(19) United States
(12) Patent Application Publication
(10) Pub. No.: US 2015/0139981 A1
(43) Pub. Date: May 21, 2015

(54) ENZYMATIC MODIFICATION OF ANTI-AQP4 AUTOANTIBODY FOR MODULATING NEUROMYELITIS OPTICA

(71) Applicant: The Regents of the University of California, Oakland, CA (US)

(72) Inventors: Alan S. Verkman, San Francisco, CA (US); Lukmanee Tradtrantip, San Francisco, CA (US)

(73) Assignee: The Regents of the University of California, Oakland, CA (US)

(21) Appl. No.: 14/402,579

(22) PCT Filed: May 21, 2013
(86) PCT No.: PCT/US2013/041955

§ 371 Date: Nov. 20, 2014

Related U.S. Application Data
(60) Provisional application No. 61/649,541, filed on May 21, 2012.

(51) Int. Cl. C07K 16/28; A61K 38/47; A61K 39/395

(52) U.S. Cl. CPC C07K 16/28; A61K 39/3955; A61K 38/47; C07K 2317/41; C07K 2317/52; A61K 2039/505
USPC 424/133.1; 530/387.3

(57) ABSTRACT

Provided herein is a method of treating neuromyelitis optica (NMO) in an animal or human subject comprising administering to the subject a composition comprising a therapeutically effective amount of an Fc region modified anli-AQP4 antibody, thereby treating the NMO in the subject, in some embodiments, the Fc region modified anti-AQP4 antibody is an anti-AQP4 antibody deglycosylated at the amino acid position Asn297. In other embodiments, the Fc region modified anti-AQP4 antibody is an anii-AQP4 antibody F(ab′)₂ fragment.

### CROSS REFERENCE TO RELATED APPLICATIONS

This application claims the priority benefit of U.S. Provisional Patent Application Ser. No. 61/649,541 filed on May 21, 2012.

### STATEMENT REGARDING FEDERALLY SPONSORED RESEARCH OR DEVELOPMENT

This invention was made with Government support under contracts EY13574, DK35124. EB00415, DK86125 and DK72517 from the National Institutes of Health. The U.S. Government has certain rights in this invention.

### TECHNICAL FIELD

The present disclosure is generally related to methods of generating modified anti-AQP4 antibodies and their use in therapeutic treatment of neuromyelitis optica.

### BACKGROUND

Neuromyelitis optica (NMO) is an inflammatory demyelinating disease primarily affecting spinal cord and optic nerve, producing paralysis and blindness (Jarius et al., (2008) Nat. Clin. Pract. Neurol. 4: 202-214; Wingerchuk et al., (2007) Lancet Neurol. 6: 805-815). Most NMO patients are seropositive for IgG autoantibodies (NMO-IgG) (Lennon et al., (2005) J. Exp. Med. 202: 473-477; Jarius & Wildemann (2010) Nat. Rev. Neurol. 6: 383-392) against aquaporin-4 (AQP4), a plasma membrane water transporting protein expressed on astrocytes throughout the central nervous system (Manley et al., (2000) Nat. Med. 6: 159-163; Nielsen et al., (1997) J. Neurosci. 17: 171-180). It is believed that NMO-IgG binding to AQP4 initiates complement- and cell-mediated cytotoxicity, resulting in inflammation, local disruption of the blood-brain barrier, and damage to oligodendrocytes and neurons. Current NMO therapies have limited efficacy and potential long-term side effects, which include immunosuppression, plasma exchange, and B-cell-depleting monoclonal antibodies (Collongues & de Seze (2011) Ther. Adv. Neurol. Disord. 4: 111-121; Cree B. (2008) Curr. Neurol. Neurosci. Rep. 8: 427-433). An open label clinical trial of an anti-complement antibody therapy (eculizumab) is in progress.

One therapeutic strategy for NMO has focused on prevention of the initiating pathogenic event of NMO-IgG binding to AQP4. In one approach, a tight-binding recombinant monoclonal antibody, derived from clonally expanded plasma blasts in NMO cerebrospinal fluid, was mutated to inhibit its effector functions for complement-dependent cytotoxicity (CDC) and antibody-dependent cell-mediated cytotoxicity (ADCC) (Tradtrantip et al., (2012) Ann. Neurol. 71: 314-322). The mutated, non-pathogenic antibody (‘aquaporumab’) competes with pathogenic NMO-IgG for AQP4 binding, preventing CDC, ADCC, and the development of NMO lesions in mouse models. In a second approach, high-throughput screening identified small-molecule blockers that bind to AQP4 and sterically prevent NMO-IgG binding to the extracellular surface of AQP4.

### SUMMARY

Briefly described, embodiments of this disclosure, among others, encompass compositions, methods of manufacture thereof, and methods of using the compositions for reducing the binding of an autoantibody to astrocytes in the central nervous system that would otherwise result in a reduction in the viability of astrocytes, neurons, and other cell types, and the pathology in neuromyelitis optica.

Neuromyelitis optica (NMO) is a demyelinating disease of the central nervous system caused by binding of pathogenic autoantibodies (NMO-IgG) to aquaporin-4 (AQP4) on astrocytes, which initiates complement-dependent cytotoxicity (CDC) and inflammation. The compositions and methods of the disclosure provide an alternative strategy involving neutralization of NMO-IgG effector function by selective heavy-chain lgG deglycosylation with Endoglycosidase S (EndoS). EndoS treatment of NMO-IgG from NMO patient sera reduced by greater than 95% CDC and antibody-dependent cell-mediated cytotoxicity (ADCC), without impairment of binding to AQP4. Cytotoxicity was also prevented by addition of EndoS after NMO-IgG binding to AQP4. The EndoS-treated, neutralized NMO-IgG competitively displaced AQP4-bound pathogenic NMO-IgG, and reduced NMO pathology in ex vivo spinal cord culture and in vivo mouse models of NMO. EndoS deglycosylation thus converts patient NMO-IgG from a pathogenic entity to a therapeutic non-pathogenic antibody, providing autologous or heterologous administration of EndoS-treated plasma to a patient as therapy of NMO.

One aspect of the present disclosure encompasses embodiments of a method of generating a therapeutic antibody effective in modulating neuromyelitis optica (NMO) when administered to an animal or human subject, the method comprising contacting an anti-AQP4 autoantibody glycosylated at the amino acid position Asn297 thereof with Endoglycosidase S under conditions whereby the Endoglycosidase S deglycosylates the autoantibody, thereby providing a therapeutic antibody capable of specifically binding to AQP4 but not capable of activating complement-dependent cytotoxicity (CDC) or antibody-dependent cell-mediated cytotoxicity (ADCC) in an animal or human subject.

### BRIEF DESCRIPTION OF THE DRAWINGS

FIGS. 1A-1D illustrate that EndoS deglycosylation of NMO-IgG prevents CDC and ADCC. FIG. 1A schematically (left) shows IgG Fc glycosylation at Asn-297, and Fab binding to AQP4. The sugar moiety at Asn-297 with EndoS cleavage site is shown to the right. Asn, asparagine; Fuc, fucose; GlcNac, N-acetylglucosamine; Man, mannose: Gal, galactose; Sial, sialic acid. FIG. 1B is a digital image showing Coomassie blue SDS-PAGE and Lens culinaris agglutinin (LCA) lectin blot of control and EndoS-treated NMO-IgG and purified IgG from NMO sera. FIG. 1C (top) is a graph showing CDC in AQP4-expressing CHO cells incubated with NMO-IgG or NMO-IgGa^{GL-} and 2% human complement, as quantified by LDH release (S.E., n=4). At bottom is a digital image showing live/dead staining of AQP4-expressing CHO cells incubated with 5 μg/mL NMO-IgG or NMO-IgG^{GL-} and 2% human complement. FIG. 1D (top) is a graph showing ADCC in AQP4-expressing CHO cells incubated with NK-cells and 20 μg/mL NMO-IgG or NMO-IgG^{GL-}, as quantified by percentage dead cells (S.E., n=4). At bottom is a digital image showing live/dead (green/red) staining.

FIGS. 2A-2C illustrate that EndoS deglycosylation of NMO serum prevents CDC and ADCC. FIG. 2A (left, top) is a graph showing CDC in AQP4-expressing CHO cells incubated with NMO serum or NMO serum^{GL-} and 2% human complement, as quantified by LDH release. At left, bottom, is a digital image showing live/dead staining. At right is a graph showing a summary of data from three NMO sera (S.E., n=6, P<0.001). FIG. 2B (top) is a graph illustrating ADCC in AQP4-expressing CHO cells incubated with NK-cells and control or EndoS-treated IgG from NMO sera (1 mg/mL), as quantified by percentage dead cells (S.E., n=5, P<0.001). At bottom is a digital image showing live/dead (green/red) staining. FIG. 2C (top) schematically illustrates that EndoS addition in situ after NMO-IgG binding to AQP4 reduces CDC. At bottom is a graph illustrating that CDC was measured by LDH release in AQP4-expressing CHO cells incubated with NMO serum for 30 minutes, then treated with EndoS for 30 minutes, followed by 2% human complement for 1 hour. (S.E., n=4, * P<0.01).

FIGS. 3A-3C illustrate that EndoS-treated NMO-IgG binds to AQP4 and competes with binding of pathogenic NMO-IgG. FIG. 3A (left) is a series of digital images showing the binding of NMO-IgG to AQP4 in CHO cells. Fluorescence micrographs show AQP4-expressing CHO cells stained for NMO-IgG or NMO-IgG^{GL-}, (red) and AQP4 (green). At right is a graph showing the binding of NMO-IgG and NMO-IgG^{GL-} showing red-to-green fluorescence ratio (R/G) as a function of NMO-IgG concentration (S.E., n=3). FIG. 3B is a series of digital images showing the binding of control and EndoS-treated NMO patient serum to AQP4 on CHO cells. FIG. 3C (top) schematically shows that EndoS-treated NMO-IgG protects against CDC caused by (untreated) NMO-IgG. The graph shows LDH release assayed in CHO cells after 1 hour incubation with indicated concentrations of NMO-IgG and NMO-IgG^{GL-} together with 2% human complement.

FIGS. 4A-4D illustrate that EndoS treatment prevents lesions in an ex vivo spinal cord slice culture model of NMO. FIG. 4A is a series of digital images showing spinal cord slice cultures exposed to 5 μg/mL NMO-IgG or NMO-IgGA^{GL-} and 5% human complement (HC). Representative GFAP and AQP4 immunofluorescence is shown after 24 hours. FIG. 4B is a bar graph showing a summary of lesion scores from experiments as in FIG. 4A (S.E., 6 slices per group, * P<0.01). FIG. 4C is a series of digital images showing slice cultures incubated with 5 μg/mL NMO-IgG, and then 30 minutes later with 20 U/mL EndoS, and 5% HC added 60 minutes later. FIG. 4D is a graph showing lesion scores (S.E., 6 slices per group, * P<0.01).

FIGS. 5A-5C illustrate that EndoS treatment prevents lesions in an in vivo mouse model of NMO. FIG. 5A is a series of digital images showing the brains of live mice injected with 0.6 μg NMO-IgG or NMO-IgG^{GL-} together with 3 μL human complement (HC). Representative GFAP. AQP4 and myelin (MBP) immunofluorescence shown at 3 days after injection. Yellow line represents needle tract. White line delimits the lesion with loss of AQP4, GFAP and myelin. FIG. 5B is a series of digital images showing higher magnification of brains injected with NMO-IgG and HC. White dashed line delimits the lesion (top). Contralateral hemispheres (non-injected) are shown (right). FIG. 5C is a series of graphs showing a summary of lesion size from experiments as in A (S.E., 4 mice per group, * P<0.01 by the non-parametric Mann-Whitney test).

FIGS. 6A-6B illustrate that IdeS cleavage of human NMO-IgG. FIG. 6A is a photograph of a Coomassie blue SDS-PAGE of control and IdeS-treated NMO-IgG (rAb-53, 5 μg, 60 minute incubation with 5 U IdeS at 37° C.). FIG. 6B is a photograph of a Coomassie blue SDS-PAGE of control and IdeS-treated antibodies (1 μg purified human antibodies incubated with 5 U IdeS for 30 minutes at 37° C.).

FIGS. 7A-7G illustrate that IdeS treatment of NMO-IgG prevents CDC and ADCC. FIGS. 7A and 7B are graphs showing CDC in AQP4-expressing CHO cells incubated for 60 minutes with control and IdeS-treated monoclonal recombinant NMO-IgGs (rAb-53, rAb-93; each 0.2-20 μg/ml) (FIG. 7A) and NMO sera (5-200 μg/ml) (FIG. 7B), each together with 2% human complement. Cytotoxicity quantified by Alamar Blue assay. (FIG. 7B inset) CDC for 3 different NMO sera (S.E., n=4). FIGS. 7C and 7D are graphs showing time course and concentration-dependence of IdeS action. In FIG. 7C, CDC was measured as in FIG. 7A for NMO-IgG rAb-93 (1 and 3 μg/ml), which was incubated for 30 minutes with indicated concentrations of IdeS prior to addition to cells. In FIG. 7D, CDC measurement was one in which NMO-IgG rAb-53 (1 and 3 μg/ml) was incubated with 1.68 U/ml IdeS for indicated times prior to addition to cells (S.E., n=4). FIGS. 7E and 7F are graphs showing that IdeS treatment of AQP4-bound NMO-IgG prevents CDC. Cells were incubated with NMO-IgGs or NMO serum for 30 min, then treated with IdeS for 30 minutes, followed by 2% human complement for 1 hour (S.E., n=4). FIG. 7G is a graph showing ADCC in AQP4-expressing CHO cells incubated with 100,000 NK cells and 0.25-10 μg/ml untreated or IdeS-treated NMO-IgG (S.E., n=4).

FIGS. 8A-8D illustrate that IdeS-treated NMO-IgG binds to AQP4. FIG. 8A provides fluorescence micrographs showing F(ab′)₂ binding (red) to AQP4 (green). FIG. 8B is a graph showing binding of NMO-IgG and NMO-IgG^{IdeS} where the red-to-green fluorescence ratio (R/G) is a function of NMO-IgG concentration (S.E., n=3). FIG. 8C provides fluorescence micrographs showing F(ab′)₂ binding (red) to AQP4 (green). FIG. 8D is a graph showing R/G at IgG concentrations of 200 and 50 μg/ml for serum 1 and 2, respectively (S.E., n=3).

FIGS. 9A-9E illustrate that IdeS-treated NMO-IgG competitively displaces pathogenic NMO-IgG, reducing cytotoxicity. FIG. 9A is a graph showing. F(ab′)2 fragments produced by IdeS cleavage of NMO-IgG competitively displace NMO-IgG. (Binding of NMO-F(ab′)₂ fragment on AQP4-expressing CHO cells incubated with NMO-IgG (1 μg/ml rAb-93) and NMO-IgG^{IdeS} or control-IgG^{IdeS}, followed by horseradish peroxidase (HRP)-conjugated anti-human IgG (Fc-specific) secondary antibody, as quantified by HRP activity assay). FIGS. 9B and 9C illustrate that IdeS-treated NMO-IgG protects against CDC caused by (untreated) NMO-IgG. Cytotoxicity was measured by Alamar Blue assay after 60 minute incubation with NMO-IgG (2 μg/ml rAb-53; 1 μlg/ml rAb-93) or NMO sera and 2% HC in AQP4-expressing cells, together with indicated concentrations of NMO-IgG^{IdeS} or NMO serum^{IdeS} (S.E., n=4). FIGS. 9C and 9D illustrate that Fc fragments generated by IdeS cleavage reduce CDC. AQP4-expressing CHO cells were incubated for 60 minutes with NMO-IgG (3 μg/ml rAb-53) and 1% human complement with different concentration of human IgG Fc fragments. FIG. 9E illustrates that Fc fragments reduce ADCC. Human IgG Fc fragments were pre-incubated with NK cells for 30 minutes at 37° C., then added together with NMO-IgG (3 μg/ml rAb-53) to AQP4-expressing CHO cells and incubated for 1 hour (S.E., n=4).

FIGS. 10A-10D illustrate that IdeS treatment of NMO-IgG prevents lesions in a mouse model of NMO. In FIG. 10A, brains of live mice were injected with 0.6 μg NMO-IgG or NMO-IgG^{IdeS} together with 3 μL human complement (HC). Representative GFAP, AQP4 and myelin (MBP) immunofluorescence at 3 days after injection. Yellow line represents needle tract. White line delimits the lesion with loss of AQP4, GFAP and myelin. The right side of FIG. 10A shows a higher magnification of brains injected with NMO-IgG and HC, where the white dashed line delimits the lesion (top). Contralateral hemispheres (non-injected) are shown (far right). FIG. 10B shows a summary of lesion size from experiments as in FIG. 10A (S.E., 4 mice per group,** p<0.01 by non-parametric Mann-Whitney test). In FIG. 10C, brains were injected with 12 μg of purified IgG from NMO serum and 48 μg of IdeS-treated IgG purified from the same NMO patient (NMO serum^{IdeS}) or a non-NMO control (control serum^{IdeS}), together with 3 μL HC. (left) Representative GFAP, AQP4 and MBP immunofluorescence at 3 days after injection. Yellow line shows the needle tract and white line delimits the lesion. FIG. 10D shows a summary of lesion size (S.E., 4 mice per group, ** p<0.01).

FIGS. 11A and 11B illustrate that EndoS efficiently cleaves NMO-IgG in mice in vivo. Mice were injected with 0.6 μg NMO-IgG and 15 minutes later at the same site with 3 μL human complement (HC) without or with 16.75 U IdeS. In FIG. 11A, representative GFAP, AQP4 and myelin (MBP) immunofluorescence is shown at 3 days after injection. Yellow line represents needle tract. White line delimits the lesion with loss of AQP4, GFAP and myelin. FIG. 11B provides a summary of lesion size from experiments as in FIG. 11A (S.E., 4 mice per group,** p<0.01 by non-parametric Mann-Whitney test).

### DETAILED DESCRIPTION

Provided herein is a method of treating neuromyelitis optica (NMO) in an animal or human subject comprising administering to the subject a composition comprising a therapeutically effective amount of an Fc region modified anti-AQP4 antibody, thereby treating the NMO in the subject. In some embodiments, the Fc region modified anti-AQP4 antibody is an anti-AQP4 antibody deglycosylated at the amino acid position Asn297. In other embodiments, the Fc region modified anti-AQP4 antibody is an anti-AQP4 antibody F(ab′)₂ fragment. Term definitions and abbreviations used in the specification and claims are as follows.

### ABBREVIATIONS

NMO, Neuromyelitis optica; AQP4 aquaporin-4; CDC, complement-dependent cytotoxicity; antibody-dependent cell-mediated cytotoxicity, ADCC; asparagine, Asn; fucose, Fuc; N-acetylglucosamine, GlcNac; mannose, Man; galactose, Gal; sialic acid, Sial.; LCA, Lens culinaris agglutinin, EndoS, Endoglycosidase S; NMO-IgG, NMO-associated autoantibody immunoglobulin G; IgG, immunoglobulin G; NMO-IgG^{GL-}, deglycosylated NMO-associated autoantibody immunoglobulin G.

### DEFINITIONS

As used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

The term “administering” refers to a method of delivering a composition of the disclosure (e.g., a deglycosylated anti-AQP4 antibody) to a patient. Such methods are well known to those skilled in the art and include, but are not limited to, oral, nasal, intravenous, intramuscular, intraperitoneal, subcutaneous, intrathecal, intradermal, or topical administration. Preferably, the therapeutic antibody of the present disclosure may be administered to an optic nerve, a ventricle of the brain, or the cerebrospinal fluid either intracranially or directly into the fluid enveloping the spinal cord. Another preferred route of administration is intravenously by such as after plasmapharesis. The route of administration can depend on a variety of factors, such as the therapeutic goals. Compositions of the disclosure may be administered on a continuous or an intermittent basis. Methods for formulating and subsequently administering therapeutic compositions are well known to those skilled in the art. See, for example. Remington, 2000, The Science and Practice of Pharmacy, 20th Ed., Gennaro & Gennaro, eds., Lippincott, Williams & Wilkins. The dose administered will depend on many factors, including the mode of administration and the formulation. Typically, the amount in a single dose is an amount that effectively reduces the level of binding of a glycosylated anti-AQP4 autoantibody to its corresponding target protein in an individual without exacerbating the disease symptoms.

The term “antibody” as used herein refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define an target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

As used herein the term “anti-AQP4 antibody F(ab′)₂ fragment” refers to an F(ab′)₂ fragment of an anti-AQP4 antibody. In some embodiments, the F(ab′)₂ fragment is an IgG fragment. The anti-AQP4 antibody F(ab′)₂ fragment F(ab′)₂ fragments of the present invention are functional in the sense that they bind to AQP4, however, these fragments do not initiate CDC or ADCC immune reactions in a host or subject. In some embodiments, the anti-AQP4 antibody F(ab′)₂ fragment is created using an IdeS

The term “antibody-dependent cell-mediated cytotoxicity” as used herein refers to the cell-killing ability of effector cells, in particular lymphocytes, which preferably require the target cell being marked by an antibody. ADCC can occur when antibodies bind to antigens on cells and the antibody Fc domains engage Fc receptors (FcR) on the surface of immune effector cells. Several families of Fc receptors have been identified, and specific cell populations characteristically express defined Fc receptors.

The term “Aquaporin 4” (AQP4) as used herein refers to a water-specific member of the Aquaporin family of water and water/glycerol transporters (Hasegawa et al., (1994) J. Biol. Chem. 269: 5497). The aquaporins are a structurally unique class of transmembrane transporter proteins where the active channel is formed at the nexus of four or more protein monomers. The aquaporins are characterized by the formation of a protein homotetramer, where each protein monomer contains a channel that is virtually independent from that of the other protein monomers (Hiroaki et al., (2006) J. Mol. Bio. 355: 628). The nexus of the AQP protein monomers is not believed to form an active channel or pore. The AQP4 water transporter is widely distributed in the human body, with particularly high concentrations in the brain, eyes, ears, muscles, lungs and kidneys (Jung et al., (1994) Proc. Nat. Acad. Sci., USA 91: 13052-13056). It is highly conserved among mammalian species, typically showing greater than 95% identity, and also has a similar biological distribution (Zardoya et al., (2001) J. Mol. Evol. 2001, 52, 391). The main role of AQP4 is generally regarded to be the regulatory water balance of the tissues in which it is localized.

The terms “astrocyte” and “oligodendrocyte” as used herein refer to a glial cell or “glial-like” cell, which includes a cell that has one or more glial-specific features, associated with a glial cell type, including a morphological, physiological and/or immunological feature specific to a glial cell (e.g. astrocytes or oligodendrocytes). For example, expression of the astroglial marker fibrillary acidic protein (GFAP) or the oligodendroglial marker 04.

The term “autoantibody” as used herein refers to an antibody manufactured by the immune system that is directed against one or more of an individual's own antigens such as an epitope of a protein, a peptide, or a non-protein epitope. Many autoimmune diseases, notably lupus erythramatosus, scleroderma, and polymyositis/dermatomyositis associated with autoantibodies. The autoantibodies of the present disclosure are directed to an epitope of the aquaporin-4 (AQP4) protein located in the astrocytes surrounding the optic and neuronal cells.

The term “complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. MAethods 202:163 (1996), may be performed. CDC is a cell-killing method that can be directed by antibodies. IgM is the most effective isotype for complement activation. IgG1 and IgG3 are also both very effective at directing CDC via the classical complement-activation pathway. Preferably, in this cascade, the formation of antigen-antibody complexes results in the uncloaking of multiple C1q binding sites in close proximity on the CH2 domains of participating antibody molecules such as IgG molecules (C1q is one of three subcomponents of complement C1). Preferably these uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a. Preferably, the complement cascade ends in the formation of a membrane attack complex, which creates pores in the cell membrane that facilitate free passage of water and solutes into and out of the cell.

In this disclosure, “comprises,” “comprising,” “containing” and “having” and the like can have the meaning ascribed to them in U.S. Patent law and can mean “includes,” “including,” and the like. “Consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

The term “effective amount” as used herein refers to that amount of a composition or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Administration of a therapeutically effective amount can be achieved with a single administration/dose or multiple administrations/dosages.

The term “endoglycosidase” as used herein especially refers to Endoglycosidase S (EndoS), a bacterial product of Streptococcus pyogenes that selectively digests asparagine-linked glycans on the heavy chain of all IgG subclasses, without action on other immunoglobulin classes or other glycoproteins. EndoS has been used to neutralize pathogenic IgG in experimental animal models of autoimmunity, including collagen-induced arthritis (Nandakumar K S. et al., Eur J Immunol 2007:37:2973-2982), immune thrombocytopenic purpura (Albert, H., et al., Proc Natl Acad Sci USA 2008:105:15005-15009), lupus erythematosus, and anti-neutrophil cytoplasmic autoantibody (ANCA)-mediated glomerulonephritis. Although EndoS has not been used in humans, a different glycosidase is in phase II clinical trials to neutralize blood group antigens to generate, ex vivo, universal blood for donation. The present disclosure provides for EndoS treatment of NMO patient serum that neutralizes the autoantibody pathogenicity without affecting AQP4 binding, and demonstrates its utility in preventing NMO pathology in cell and mouse models.

As used herein, the term “Fc region modified anti-AQP4 antibody” refers to an antibody that selectively binds to an AQP4 moiety and contains a modified or deleted Fc region, which modification renders the antibody with a reduced ability to activate or initiate CDC and/or ADCC mediated immune reactions (such as CDC and/or ADCC mediated immune reactions against astrocytes). In some embodiments, the Fc region modified anti-AQP4 antibody is deglycosylated at the amino acid position Asn297. In other embodiments, the Fc region modified anti-AQP4 antibody is an anti-AQP4 antibody F(ab′)₂ fragment. In preferred embodiments, the Fc region modified anti-AQP4 antibody competes with an un-modified anti-AQP4 antibody for binding to an AQP4 moiety, and thereby reduces the binding of the un-modified anti-AQP4 antibody to AQP4.

The term “glycosylation” as used herein refers to the attachment of glycans at specific locations along the polypeptide backbone and is usually of two types: O-linked oligosaccharides are attached to serine or threonine residues while N-linked oligosaccharides are attached to asparagine residues when they are part of the sequence Asn-X-Ser/Thr, where X can be any amino acid except proline. The structures of N-linked and O-linked oligosaccharides and the sugar residues found in each type are different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein.

The term “glycosylation site” as used herein refers to a location on a polypeptide that has a glycan chain attached thereto, such as, but not limited to, the Asn297 position of the Fc region of an immunoglobulin G. The “site” may be an amino acid side-chain, or a plurality of side-chains (either contiguous in the amino acid sequence or in cooperative vicinity to one another to define a specific site associated with at least one glycosylation chain). The term “glycosylation site” as used herein further refers to a combination of a region of a polypeptide, and a region of a glycan chain attached to the polypeptide.

The term “IdeS” refers herein an IgG-degrading enzyme of Streptococcus pyogenes, also called Mac 1, which efficiently cleaves human IgGs of all subclasses without effect on other antibody classes or proteins. Compared to EndoS. IdeS: (i) has greater rate of IgG cleavage compared to the rate of EndoS deglycosylation; (ii) produces antibody fragments with zero residual effector function; and (iii) generates free Fc fragments that block Fc receptors on phagocytes. IdeS has shown efficacy in rodent models of experimental arthritis caused by anti-collagen antibodies (Nandakumar et al., 2007), idiopathic thrombocytopenic purpura caused by anti-platelet antibodies (Johansson et al., 2008), and glomerulonephritis caused by anti-glomerular basement membrane antibodies (Yang et al., 2010).

The term “immobilized on a solid support” as used herein refers to such as an Endoglycosidase S, an antibody, an antigen-binding fragment, and the like attached to a substance at a particular location in such a manner that the system containing the immobilized entity may be subjected to washing or other physical or chemical manipulation without being dislodged from that location. A number of solid supports and means of immobilizing polypeptide-containing molecules to them are known in the art. For example, and not intended to be limiting, immobilization onto a solid support may be by directly chemically cross-linking a molecular species to an underlying support material. Alternatively, and especially useful for linking an immunoglobulin to a solid support as used in the methods of the disclosure, an immunoglobulin-binding agent such as an anti-immunoglobulin antibody may be attached thereto. It is understood that a solid support can be such as, but not limited to, beads, plastic surfaces, or any other surface to which an immunoglobulin-binding agent can be bound such as, but not intended to be limiting, SEPHAROSE®, agarose, polyacrylamide and the like. Of particular usefulness in the methods of the disclosure is an Endoglycosidase S attached to a solid support that may be contacted in a batch-wise or continuous flow system with a composition containing the antibody species to be deglycosylated.

The term “isolated” antibody as used herein refers to an antibody identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. In advantageous embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “modulates” as used herein refers to a change in the level of activity, or the amount of, a constituent of an animal or human, an organ, thereof, or a cell or isolated cell thereof. For example, but not intended to be limiting, “modulate” may refer to an decrease in the binding of an antibody such as glycosylated anti-AQP4 antibody due to competition for the specific target epitope by deglycosylated antibody according to the present disclosure. Alternatively, “modulate” may refer to a decrease in the extent of loss of neuronal activity or viability due to the binding of the glycosylated autoantibody to AQP4. As used herein, “modulating” a neurological disorder can refer to reducing the severity of one or more symptoms, eliminating all symptoms, any level of symptoms there between, or inhibiting the onset of the neurological disorder.

The term “neuromyelitis optica” (NMO) as used herein refers to a neurological disorder also known as Devic's syndrome in Western countries and as opticopinal multiple sclerosis in Asia. NMO is regarded as a severe variant of multiple sclerosis (MS), and accounts for 30% of MS cases occurring in Asians. In North America, non-Caucasians represent a higher frequency of patients with NMO than the frequency of those with classical MS. The characteristic inflammatory demyelinating lesions of NMO selectively and repeatedly affect the optic nerves and the spinal cord, thereby causing both blindness and paralysis.

The terms “organism,” “host,” and “subject” as used herein refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). “Subject” may also be a cell, a population of cells, a tissue, an organ, or an organism, preferably human, and constituents thereof.

The term “pharmaceutically acceptable carrier” as used herein refers to a diluent, adjuvant, excipient, or vehicle with which a heterodimeric probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the heterodimeric probe and pharmaceutically acceptable carriers can be sterile. Water is a useful carrier when the heterodimeric probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “plasmapharesis” as used herein refers to methods and extracorporeal systems for apheresis (i.e., the process of withdrawing blood from an individual, removing components from the blood, and returning the blood, or blood depleted of one or more components such as, but not limited to, antibodies, to the individual. Antibodies removed can be of any class, e.g., IgG (such as IgG1, IgG2, IgG3, and IgG4), IgM, IgD, IgA, or IgE antibodies as are known in the art (see, for example, U.S. Pat. Nos. 4,708,713; 5,258,503; 5,386,734; and 6,409,696).

The term “protein” as used herein refers to a large molecule composed of one or more chains of amino acids in a specific order. The order is determined by the base sequence of nucleotides in the gene coding for the protein. Proteins are required for the structure, function, and regulation of the body's cells, tissues, and organs.

The term “substantially pure” as used herein means an object species is the predominant species present (i.e., on a molar basis it is more abundant than any other individual species in the composition), and preferably a substantially purified fraction is a composition wherein the object species comprises at least about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

The terms “treating” and “treatment” as used herein refer generally to obtaining a desired pharmacological and/or physiological effect. The effect may be prophylactic in terms of preventing or partially preventing a disease, symptom or condition thereof such as neuromyelitis optica, and/or may be therapeutic in terms of a partial or complete cure of a disease, condition, symptom or adverse effect attributed to the disease. The term “treatment” as used herein covers any treatment of neuromyelitis optica in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions.

The term “treatment” as used herein particularly refers to the administration of a compound in an amount sufficient to, alleviate, ameliorate, or delay the progress of one or more symptoms or conditions associated with neuromyelitis optica (NMO). In some embodiments, treating a subject having NMO with an Fc modified anti-AQP4 antibody of the present invention results in a decrease in the amount of NMO induced lesions in the subject as compared to a control subject. In these embodiments, the lesions can be reduced by approximately 95%, 80-90%, 50-60%, 30-40%, 10-20% or 5%.

The term “treatment” as used herein refers to both therapeutic treatment and prophylactic or preventative measures. Those in need of treatment include those already with the disorder as well as those in which the disorder is to be prevented.

### Description

Provided herein is a method of treating neuromyelitis optica (NMO) in an animal or human subject comprising administering to the subject a composition comprising a therapeutically effective amount of an Fc region modified anti-AQP4 antibody, thereby treating the NMO in the subject. In some embodiments, the Fc region modified anti-AQP4 antibody is an anti-AQP4 antibody deglycosylated at the amino acid position Asn297. In other embodiments, the Fc region modified anti-AQP4 antibody is an anti-AQP4 antibody F(ab′)₂ fragment.

The present disclosure encompasses embodiments of methods for reducing the onset or intensity of autoimmune neuropathy mediated by antibodies directed to the protein aquaporin-4 (AQP4). This membrane-bound polypeptide is found in the membranes of astrocyte cells that surround neuronal cells. An anti-AQP4 IgG autoantibody, when bound to its target AQP4 initiates complement and cell-mediated cytotoxicity and progressive destruction of the neuronal cells. This will result in deterioration of the optic and spinal nerves, leading to blindness and paralysis.

It has now been surprisingly demonstrated that either removal of a glycan moiety attached to the anti-AQP4 autoantibody or removal of the Fc region of the anti-AQP4 autoantibody eliminates the ability of the IgG to trigger complement and cell-mediated cytotoxicity while retaining the affinity for the target membrane-bound protein AQP4. The deglycosylated and Fc region anti-AQP4 autoantibody deletion products are collectively referred to herein as an Fc region modified anti-AQP4 antibody. Accordingly, the Fc region modified anti-AQP4 antibody can compete with the glycosylated form for the target site and, once bound to the AQP4, prevents the cytoxicity that would otherwise be induced.

The method of the disclosure, therefore, provides an Fc region modified anti-AQP4 antibody by either removing the glycan at the Asn297 position within the Fc region of the antibody using Endoglycosidase S or removing the Fc region using an IdeS enzyme. It is contemplated that several different routes may be taken for modification of the anti-AQP4 antibody. In some embodiments of the disclosure, serum from a patient known to be producing the anti-AQP4 autoantibodies may be contacted with the EndoS and/or IdeS, which is then allowed to cleave off the glycan or the Fc region from the IgG polypeptide. In one embodiment, the EndoS and/or IdeS may be mixed in solution with the serum and, after a suitable reaction time; the EndoS and/or IdeS may be removed from the serum by a method such as passage through an anti-EndoS and/or IdeS affinity column. The serum now comprising an Fc region modified anti-AQP4 antibody, including an IgG Fc region modified anti-AQP4 antibody, but with the EndoS and/or IdeS substantially or completely separated therefrom, may be returned to the patient as a source of autologous an Fc region modified anti-AQP4 antibody which will not induce an immune response thereto. The newly modified antibody may then compete with the glycosylated/Fc containing form of the autoantibody, displacing the latter, and therefore blocking the induction of complement and cell-mediated cytotoxicity events.

While it is possible to remove serum from the patient to be treated, it is also contemplated that the serum may be continuously depleted of unmodified (i.e., natural) anti-AQP4 antibody by treating the patient with plasmapharesis, passing the plasma through a column of EndoS and/or IdeS attached to a beaded solid support or through an EndoS and/or IdeS-bound membrane system.

Plasma or serum from the animal or human subject to be treated can be usefully returned to the patient by an intravenous route. If returned to the same subject that served as the source of the serum, the method provides autologous antibodies that will not induce an adverse immune response, especially if the treatment is applied over a prolonged period. An acute application of the treated serum or plasma to a heterologous subject may offer immediate advantages in the reduction of unmodified (i.e., natural) anti-AQP4 antibody binding, but its use in such a context is limited due to undesirable immune reactions being triggered.

In other embodiments of the methods for preparing a Fc region modified anti-AQP4 antibody of the disclosure, it is contemplated that the IgG fraction of a serum may be isolated by such as an affinity column specific for IgG, or more specifically, that the anti-AQP4 autoantibody fraction be isolated by AQP4-specific affinity chromatography, followed by reacting the EndoS and/or IdeS with the isolated IgG fraction, and subsequently administering the Fc region modified anti-AQP4 antibody to an animal or human subject in need thereof. The treated Fc region modified anti-AQP4 antibody fraction may be administered to the recipient subject by means other than intravenous, including, but not limited to, subdural delivery, directly to an optic nerve, directly to the brain tissue or the ventricles thereof, lumbar puncture, to the cerebral-spinal fluid and the like, providing a localized site of administration.

While it is recognized that there are advantages to providing autologous Fc region modified anti-AQP4 antibody to the recipient subject, it is further recognized that a therapeutic composition comprising an isolated Fc region modified anti-AQP4 antibody is advantageous for administering heterologously to a recipient animal or human subject. Accordingly, the present disclosure provides methods for substantially purifying an antibody from a source such as serum using such methods as affinity chromatography, wherein, for example, an IgG fraction may be isolated by such as a Protein-A column preceding an affinity column selectively binding to immobilized AQP4. It is then possible to wash the antibody bound to the immobilized AQP4 with a solution comprising EndoS and/or IdeS to modify the antibody before it is eluted from the AQP4 affinity column. It is further considered that one of skill in the art will be able to apply any technique that can provide a substantially purified anti-AQP4 antibody that may be treated with EndoS and/or IdeS. After treatment with the EndoS and/or IdeS, the EndoS and/or IdeS and the Fc region modified anti-AQP4 antibody may be fractionated to remove the EndoS and/or IdeS before administration to an animal or human subject. Removal of the EndoS and/or IdeS is desirable to avoid deglycosylation and/or Fc removal and functional inactivation of antibodies other than the ani-AQP4 autoantibody species.

The present disclosure, therefore, provides methods for the selective enzymatic deglycosylation or Fc removal of patient NMO-IgG to neutralize its effector function without affecting its binding to AQP4, thus converting a pathogenic antibody into a therapeutic blocking antibody. Glycosylation of a conserved asparagine (Asn-297) on the CH2 domain of IgG heavy chains is essential for antibody effector functions (Jefferis et al., (2008) Immunol Rev. 163: 59-76). Modification of the Fc glycan can alter IgG conformation and reduces the Fc affinity for binding of complement protein C1q and effector cell receptor FcR. Complete removal of the Fc glycan also abolishes CDC and ADCC.

The results of the present disclosure show that EndoS and/or IdeS treatment abolished NMO-IgG effector functions, preventing complement- and cell-mediated cytotoxicity without affecting binding to AQP4. Pathogenic NMO autoantibodies were thus converted enzymatically into non-pathogenic blocking antibodies, as Fc region modified anti-AQP4 NMO-IgG antibody competes with pathogenic NMO-IgG for binding to AQP4. EndoS and/or IdeS neutralized NMO-IgG effector function in multiple NMO sera, preventing CDC and ADCC in cell cultures, and the development of NMO pathology in mouse models of NMO. EndoS and/or IdeS was effective as well in neutralizing AQP4-bound NMO-IgG in situ, as cytotoxicity and NMO pathology were prevented when EndoS and/or IdeS was added after NMO-IgG was fully bound to cell surface AQP4. Competition of EndoS and/or IdeS-treated NMO-IgG with untreated, pathogenic NMO-IgG was also shown, similar to when unrelated monoclonal recombinant NMO antibodies compete for binding to AQP4 with polyclonal NMO-IgG in NMO patient sera (Tradtrantip L, et al., Ann Neurol 2012:71:314-322). The large size of NMO-IgG compared to AQP4 is the molecular basis of this steric competition. The findings of the present disclosure support the usefulness of IgG-selective endoglycosidases and proteinases, and in particular Endoglycosidase S and IdeS, for NMO therapy.

EndoS is the only known endoglycosidase that selectively hydrolyzes glycans on IgG, without affecting IgA, IgM or other glycoproteins (Collin M, and Olsen A., EMBO 2001; 20:3046-3055). Other endoglycosidases, such as EndoF₁₋₃ from Elizabethkingia meningoseptica, or EndoE from Enterococcus faecalis, have broad spectra of activity and can cleave glycans on many glycoproteins. Intravenous injection of 10 μg EndoS in mice prevented the development of lupus-like disease in a model of spontaneous lupus without observed toxicity (Nandakumar K S, et al., Eur J Immunol 2007; 37:2973-2982). Repeated injections of EndoS in rabbits produced an anti-EndoS antibody response, but did not alter EndoS pharmacokinetics or endoglycosidase activity. Id. “IdeS” is an IgG-degrading enzyme of Streptococcus pyogenes, also called Mac 1, which efficiently cleaves human IgGs of all subclasses without effect on other antibody classes or proteins. Compared to EndoS. IdeS: (i) has greater rate of IgG cleavage compared to the rate of EndoS deglycosylation; (ii) produces antibody fragments with zero residual effector function; and (iii) generates free Fc fragments that block Fc receptors on phagocytes. IdeS has shown efficacy in rodent models of experimental arthritis caused by anti-collagen antibodies (Nandakumar et al., 2007), idiopathic thrombocytopenic purpura caused by anti-platelet antibodies (Johansson et al., 2008), and glomerulonephritis caused by anti-glomerular basement membrane antibodies (Yang et al., 2010).

Intravenous EndoS and/or IdeS administration in humans is predicted to neutralize IgG globally, but likely cause an immune response, precluding chronic administration, but potentially allowing its use in acute disease exacerbations. Intrathecal or retro-orbital administration of EndoS and/or IdeS can minimize these concerns, targeting EndoS and/or IdeS to NMO lesions in spinal cord and optic nerve.

One aspect of the present disclosure encompasses embodiments of a method of generating a therapeutic antibody effective in modulating neuromyelitis optica (NMO) when administered to an animal or human subject, the method comprising contacting an anti-AQP4 autoantibody glycosylated at the amino acid position Asn297 thereof with Endoglycosidase S under conditions whereby the Endoglycosidase S deglycosylates the autoantibody, thereby providing a therapeutic antibody capable of specifically binding to AQP4 but not capable of activating complement-dependent cytotoxicity (CDC) or antibody-dependent cell-mediated cytotoxicity (ADCC) in an animal or human subject.

In embodiments of this aspect of the disclosure, the anti-AQP4 autoantibody glycosylated at the amino acid position Asn297 thereof can be an isolated immunoglobulin G.

In embodiments of this aspect of the disclosure, the method can further comprise isolating the anti-AQP4 autoantibody glycosylated at the amino acid position Asn297 thereof from the serum of an animal or human subject.

In embodiments of this aspect of the disclosure, the step of isolating the anti-AQP4 autoantibody from the serum of an animal or human subject can comprise fractionating the serum with an affinity column capable of specifically binding immunoglobulin G or an anti-AQP4 autoantibody, and eluting the anti-AQP4 autoantibody therefrom.

In embodiments of this aspect of the disclosure, the step of contacting the anti-AQP4 autoantibody glycosylated at the amino acid position Asn297 thereof with the Endoglycosidase S can comprise contacting a composition comprising the glycosylated autoantibody with Endoglycosidase S attached to a solid support. In some embodiments of this aspect of the disclosure, the solid support can be a beaded material or a membrane.

In some embodiments of this aspect of the disclosure, the Endoglycosidase S can be attached to the solid support material of a chromatography column.

In embodiments of this aspect of the disclosure, the method can further comprise the steps of subjecting the serum isolated from the animal or human subject to plasmapharesis, whereby the anti-AQP4 autoantibody is contacted with the Endoglycosidase S resulting in deglycosylation of the autoantibody at the amino acid position Asn297 thereof; and returning the serum to the animal or human subject.

In embodiments of this aspect of the disclosure, the method can comprise the steps of administering to a subject animal or human an amount of Endoglycosidase S effective in deglycosylating an anti-AQP4 autoantibody in the subject animal or human. In these embodiments, the Endoglycosidase S can be delivered to the subject animal or human subject intrathecally, intravenously, subdurally, directly to an optic nerve, or to the cerebrospinal fluid

Still another aspect of the present disclosure encompasses embodiments of a method of reducing the binding of an anti-AQP4 autoantibody to AQP4 of a cell of an animal or human subject, the method comprising administering to the subject an amount of an anti-AQP4 autoantibody deglycosylated at the amino acid position Asn297 thereof, thereby competitively inhibiting the binding of a glycosylated anti-AQP4 autoantibody to the AQP4 of the cell.

Another aspect of the present disclosure encompasses embodiments of a method of modulating neuromyelitis optica (NMO) in an animal or human subject comprising administering to the subject a therapeutically effective amount of an anti-AQP4 autoantibody deglycosylated at the amino acid position Asn297 thereof, whereby the deglycosylated autoantibody competes with a glycosylated variant of anti-AQP4 autoantibody of the animal or human subject for binding to the target AQP4 protein of astrocytes, thereby reducing the onset or extent of a complement-dependent cytotoxicity (CDC) and antibody-dependent cell-mediated cytotoxicity (ADCC) response directed against the astrocytes.

In embodiments of this aspect of the disclosure, the deglycosylated autoantibody can be generated by any of the aforementioned methods of the disclosure.

In some embodiments of this aspect of the disclosure, the deglycosylated autoantibody can be obtained by administering to the subject an amount of Endoglycosidase S effective in deglycosylating the anti-AQP4 autoantibody at the amino acid position Asn297 thereof. In these embodiments, the Endoglycosidase S can be administered to an animal or human subject intrathecally, intravenously, subdurally, directly to an optic nerve, to the cerebrospinal fluid, or during plasmapharesis.

In embodiments of this aspect of the disclosure, the deglycosylated autoantibody can be a component of a pharmaceutically acceptable composition administered to the animal or human subject.

In embodiments of this aspect of the disclosure, the deglycosylated autoantibody can be an autologous antibody or a heterologous antibody of the animal or human subject.

In embodiments of this aspect of the disclosure, the deglycosylated autoantibody can be provided to the animal or human subject by deglycosylating the anti-AQP4 autoantibody during plasmapharesis treatment of the animal or human subject.

In embodiments of this aspect of the disclosure, the therapeutically effective amount of the anti-AQP4 autoantibody deglycosylated at the amino acid position Asn297 thereof can be administered to an animal or human subject intrathecally, intravenously, subdurally, directly to an optic nerve, to the cerebrospinal fluid, or during plasmapharesis.

Still another aspect, therefore, of the present disclosure encompasses embodiments of an isolated therapeutic antibody effective in modulating neuromyelitis optica (NMO), where the therapeutic antibody can be an anti-AQP4 immunoglobulin G deglycosylated at the amino acid position Asn297 thereof.

Another aspect of the present disclosure encompasses embodiments of a pharmaceutically acceptable composition comprising a therapeutically effective amount of an isolated therapeutic antibody effective in modulating neuromyelitis optica (NMO), wherein said therapeutic antibody is an anti-AQP4 immunoglobulin G deglycosylated at the amino acid position Asn297 thereof.

Yet another aspect of the present disclosure encompasses embodiments of a composition comprising an isolated therapeutic antibody effective in modulating neuromyelitis optica (NMO), where the therapeutic antibody is an anti-AQP4 immunoglobulin G deglycosylated at the amino acid position Asn297 thereof, and wherein the antibody is non-pathogenic.

In embodiments of this aspect of the disclosure, the composition can be an isolated serum.

In embodiments of this aspect of the disclosure, the composition can further comprise a pharmaceutically acceptable carrier.

Another aspect of the present disclosure encompasses embodiments of a method of generating a therapeutic antibody effective in modulating neuromyelitis optica (NMO) when administered to an animal or human subject, the method comprising contacting an anti-AQP4 autoantibody with IdeS under conditions whereby the IdeS removes the Fc region of the autoantibody, thereby providing a therapeutic antibody capable of specifically binding to AQP4 but not capable of activating complement-dependent cytotoxicity (CDC) or antibody-dependent cell-mediated cytotoxicity (ADCC) in an animal or human subject.

Data provided below in the Examples indicates that IdeS neutralized NMO-IgG pathogenicity, abolishing CDC and ADCC effector functions, and yielding therapeutic F(ab′)2 and Fc fragments that blocked NMO-IgG binding to AQP4 and Fcγ receptors, respectively. IdeS efficiently cleaved both free and AQP4-bound NMO-IgG, without effect on AQP4 binding of the product NMO-F(ab′)2 fragment. It was further determined that efficient cleavage of AQP4-bound NMO-IgG was accomplished when administered by intracerebral injection. IdeS cleavage of NMO-IgG was sufficiently rapid to prevent NMO lesions in mouse brain after NMO-IgG was already bound to AQP4, in which IdeS and complement were coadministered 15 minutes after NMO-IgG.

Accordingly, in these embodiments, a method of treating neuromyelitis optica (NMO) in an animal or human subject is provided that comprises administering to the subject a composition comprising a therapeutically effective amount of an Fc region modified anti-AQP4 antibody, thereby treating the NMO in the subject, wherein the Fc region modified anti-AQP4 antibody is an anti-AQP4 antibody F(ab′)₂ fragment. In some embodiments, the anti-AQP4 antibody, and thus the anti-AQP4 antibody F(ab′)₂ fragment, is an immunoglobulin G antibody. The anti-AQP4 antibody F(ab′)₂ fragment can be created by treatment of an anti-AQP4 antibody with an IdeS enzyme.

It should be understood that the anti-AQP4 antibody F(ab′)₂ fragment or the IdeS can be administered to the subject via any method known to those of ordinary skill in the art. In some embodiments, the anti-AQP4 antibody F(ab′)₂ fragment or the IdeS are administered to the subject intrathecally, intravenously, subdurally, directly to an optic nerve, to the cerebrospinal fluid, or during plasmapharesis. Preferably, in these embodiments, the IdeS is administered to the subject in an amount effective to create the Fc region modified anti-AQP4 antibody in vivo.

As mentioned above, IdeS cleavage may be accomplished by therapeutic apheresis in which patient blood is passed over surface-immobilized IdeS. Alternatively, notwithstanding potential concerns about immunogenicity, IdeS might be administered by intravenous injection, or by intrathecal or retro-orbital routes to target NMO lesions with minimal systemic exposure.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%. ±7%, ±8%, ±9%, or ±10%, or more of the numerical value(s) being modified. Unless indicated otherwise, parts are parts by weight, temperature is in ° C., and pressure is at or near atmospheric. Standard temperature and pressure are defined as 20° C. and 1 atmosphere.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims. All patents, patent applications, and publications referenced herein are incorporated by reference in their entirety for all purposes.

### EXAMPLES

### Example 1

### Cell Culture and Antibodies for EndoS Treatment

Chinese hamster ovary (CHO) cells stably expressing human M23-AQP4 were generated as described in Crane et al., (2011) J. Biol. Chem. 286: 16516-16524, incorporated herein by reference in its entirety, and cultured at 37° C. in 5% CO₂/95% air in F-12 Ham's Nutrient mix medium supplemented with 10% fetal bovine serum, 200 μg/mL geneticin (selection marker), 100 units/mL penicillin and 100 μg/mL streptomycin. Recombinant monoclonal NMO antibody rAb-53 (referred to as NMO-IgG) was generated from a clonally expanded plasma blast population from cerebrospinal fluid (CSF) of an NMO patient, as described and previously characterized (Crane et al., (2011) J. Biol. Chem. 286: 16516-16524; Bennett et al., (2009) Ann. Neurol. 66: 617-629).

NMO serum was obtained from NMO-IgG seropositive individuals who met the revised diagnostic criteria for clinical disease (Wingerchuk et al., (2006) Neurology. 66: 1485-1489). Non-NMO human serum was used as control. For some studies IgG was purified from NMO or control serum using a Melon Gel IgG Purification Kit (Thermo Fisher Scientific, Rockford, Ill.) and concentrated using Amicon Ultra Centrifugal Filter Units (Millipore, Billerica, Mass.).

### Example 2

### EndoS Treatment

EndoS was purchased from Bulldog Bio Inc. (Rochester, N.Y.). NMO-IgG or NMO serum (or control IgG/serum) was incubated with EndoS (1 unit per 1-10 μg IgG) for up to 1 hour at 37° C. Treated antibody is referred to as NMO-IgG^{GL-}. Treated NMO serum is referred to as NMO serum^{GL-}. EndoS treatment efficiency was assessed by 10% sodium dodecyl sulfate polyacrylamide gel electrophoresis (SDS-PAGE) followed by staining with Coomassie Blue or Lens culinaris agglutinin (LCA)-lectin blot analysis, as described in Albert et al., (2008) Proc. Natl. Acad. Sci. USA 105: 15005-15009.

### Example 3

### NMO-IgG Binding

Cells were grown on glass coverslips for 24 hours. After blocking with 1% BSA in PBS, cells were incubated with NMO-IgG or NMO serum (control or EndoS-treated) for 1 hour at room temperature. Cells were washed with PBS and incubated with Alexa-Flour 555 goat anti-human IgG secondary antibody (1:200, Invitrogen). For AQP4-immunostaining, cells were fixed in 4% paraformaldehyde (PFA) and permeabilized with 0.2% Triton-X. Rabbit anti-AQP4 antibody (1:200, Santa Cruz Biotech) was added followed by Alexa Fluor-488 goat anti-rabbit IgG secondary antibody (1:200, Invitrogen) for quantitative ratio image analysis, as described in Crane et al., (2011) J. Biol. Chem. 286: 16516-16524.

### Example 4

### Testing of Complement- and Cell-Mediated Cytotoxicity Mediated by NMO-IgG

For assay of CDC, cells were incubated for 60 minutes at 37° C. with NMO-IgG or NMO serum (control or EndoS-treated) with 2% human complement (Innovative Research, Novi, Mich.). In some experiments NMO-IgG was added 30 minutes before EndoS addition, followed 60 minutes later by complement. Cytotoxicity was measured by LDH release assay (Promega, Madison, Wis.) or live/dead cell staining, as described in Phaun et al., (2012) J. Biol. Chem. 287: 13829-13839. Calcein-AM and ethidium-homodimer (Invitrogen) were added to stain live cells green and dead cells red. For assay of ADCC, NK-92 cells expressing CD16 (Conkwest, San Diego, Calif.) were used as the effector cells. The AQP4-expressing CHO cells were incubated for 2 hours at 37° C. with NMO-IgG and effector cells at an effector:target cell ratio of 20:1, followed by live-dead cell staining.

### Example 5

### EndoS Deglycosylation of NMO-IgG Prevents Cytotoxicity

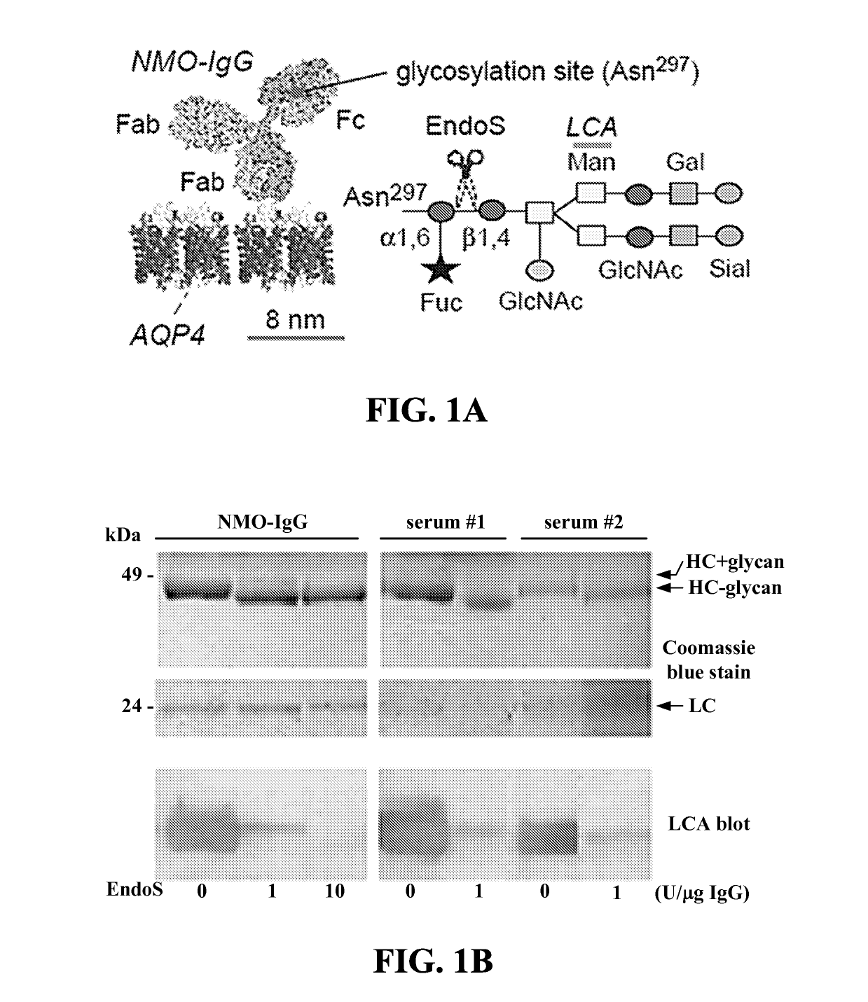

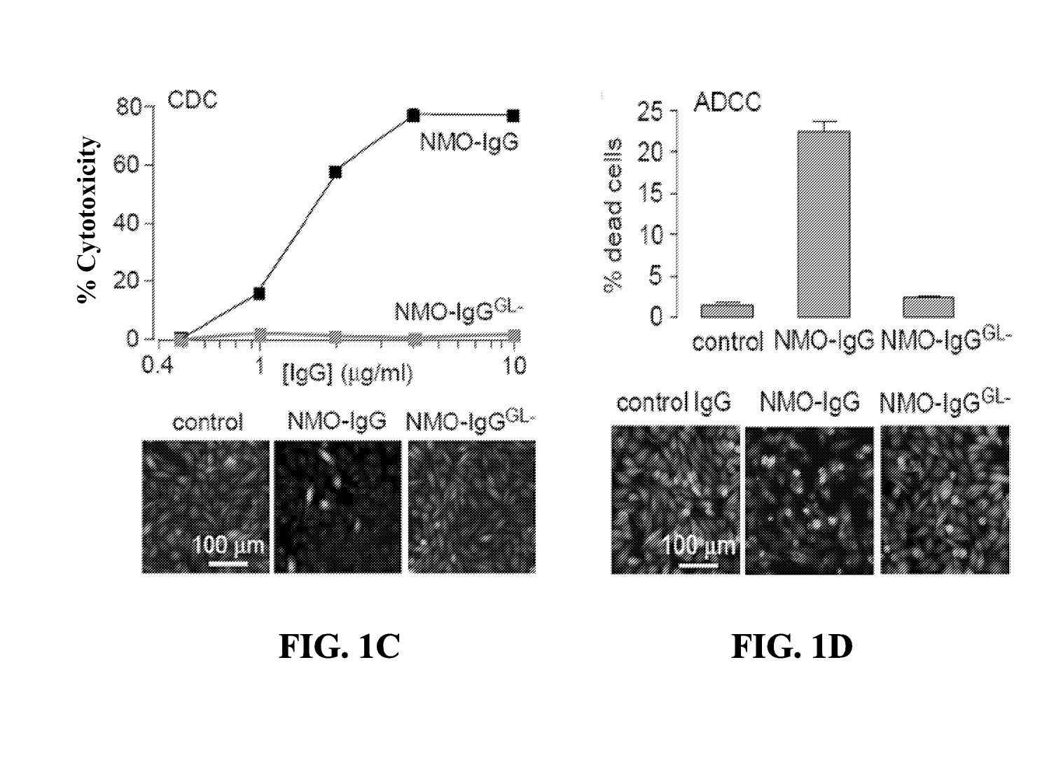

FIG. 1A diagrams N-linked glycosylation on asparagine-297 on the CH2 domain of both IgG heavy chains. EndoS selectively cleaves the β1-4 linkage between two N-acetylglucosamines located in the conserved core of the N-linked glycan of IgG. FIG. 1B shows SDS-PAGE stained with Coomassie blue (top) and lectin blot (bottom) of control and EndoS-treated NMO-IgG or IgG from NMO patient sera. Lectin blot analysis using Lens culinaris agglutinin (LCA) recognizes α-linked mannose residues, showing loss of reactivity with removal of the glycan moiety. EndoS treatment resulted in IgG deglycosylation as seen by reduced molecular size by approximately 3 kDa of the heavy chains and loss of LCA signal. Deglycosylation was near complete by 60 minutes with 10 units EndoS per 1 μg IgG.

The major effector functions of NMO-IgG were abolished by EndoS treatment. FIG. 1C shows loss of CDC in AQP4-expressing cells by LDH release (top) and live/dead staining (bottom) assays. CDC was compared in cells exposed to control or EndoS-treated NMO-IgG, together with human complement. EndoS treatment prevented CDC even at high antibody concentration. FIG. 1D shows loss of ADCC in AQP4-expressing cells by live/dead staining. ADCC was compared in cells exposed to control or EndoS-treated NMO-IgG together with human NK-cells.

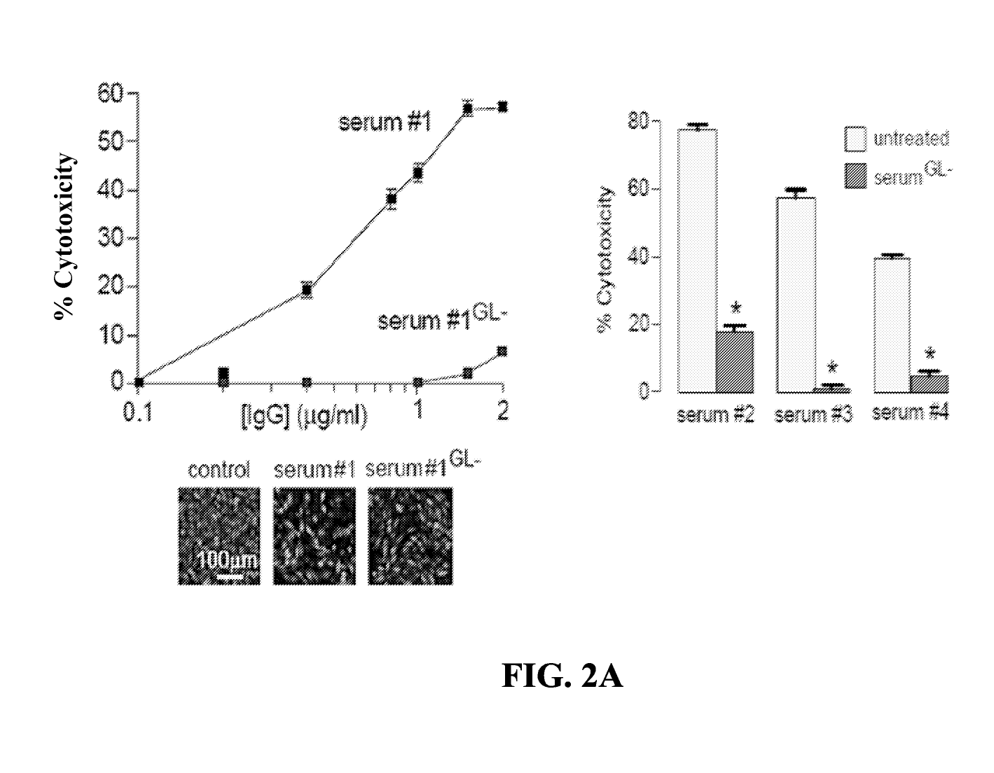

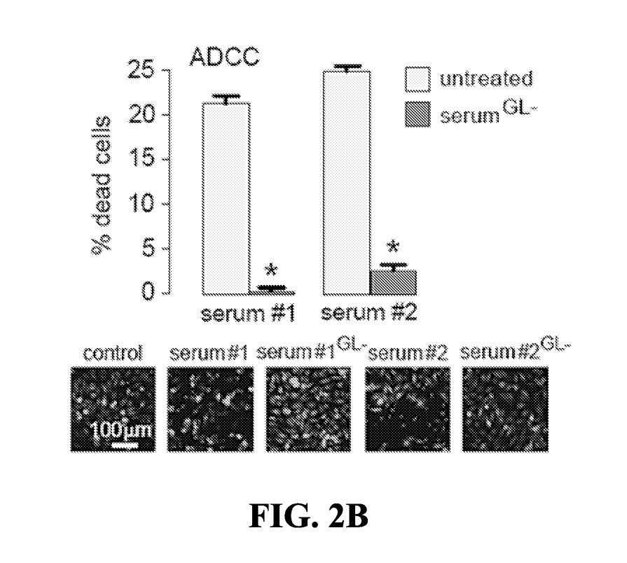

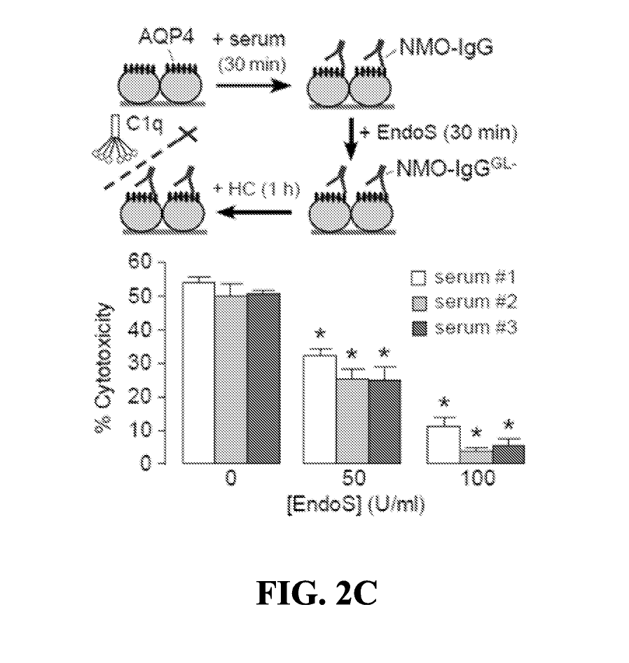

Cytotoxicity measurements were also done using NMO sera, which contain a complex, polyclonal mixture of NMO antibodies. FIG. 2A (left) shows prevention of CDC by EndoS treatment in a NMO serum specimen, as revealed by LDH release (top) and live/dead staining (bottom). Data for three additional sera from different NMO patients are summarized in FIG. 2A (right). EndoS treatment (1 unit per μg IgG for 60 minutes) greatly reduced CDC. FIG. 2C shows that EndoS treatment prevented ADCC in two NMO sera tested.

Having demonstrated that pre-treatment of NMO-IgG and NMO sera with EndoS prevents cytotoxicity, we tested whether post-treatment, after antibody binding to AQP4, is effective. For these studies AQP4-expressing cells were pre-incubated for 30 minutes with NMO-IgG, then EndoS was added, followed 30 minutes later by complement. FIG. 2C shows that post-treatment with EndoS was effective, indicating EndoS deglycosylation of AQP4-bound NMO antibody can occur in situ.

### Example 6

### EndoS Deglycosylated NMO-IgG Competes with Binding of Pathogenic NMO-IgG to AQP4

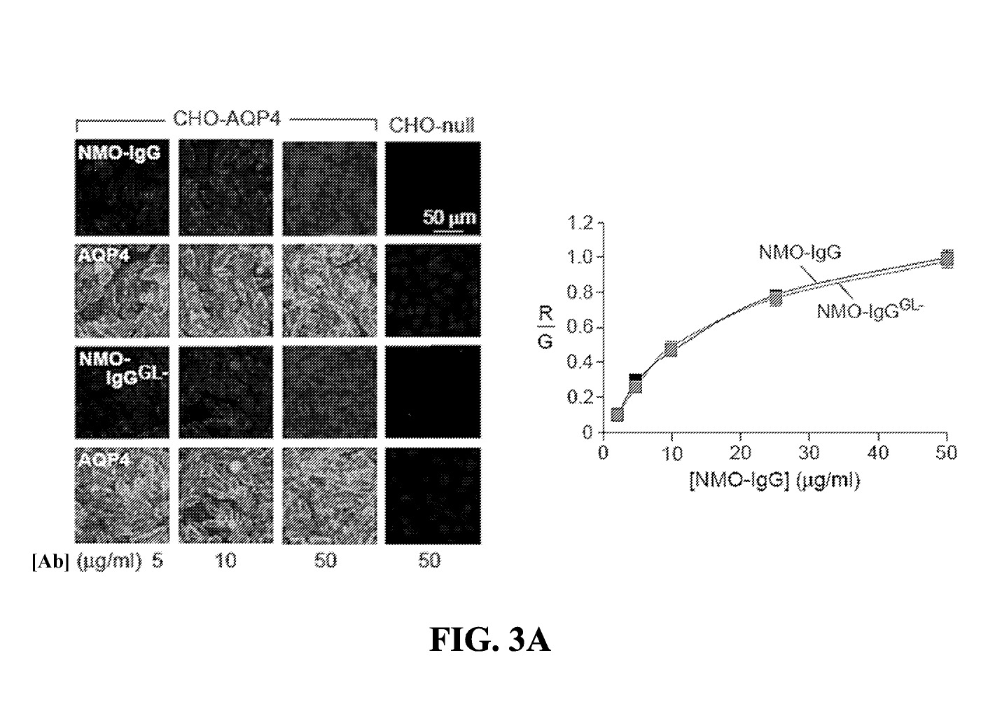

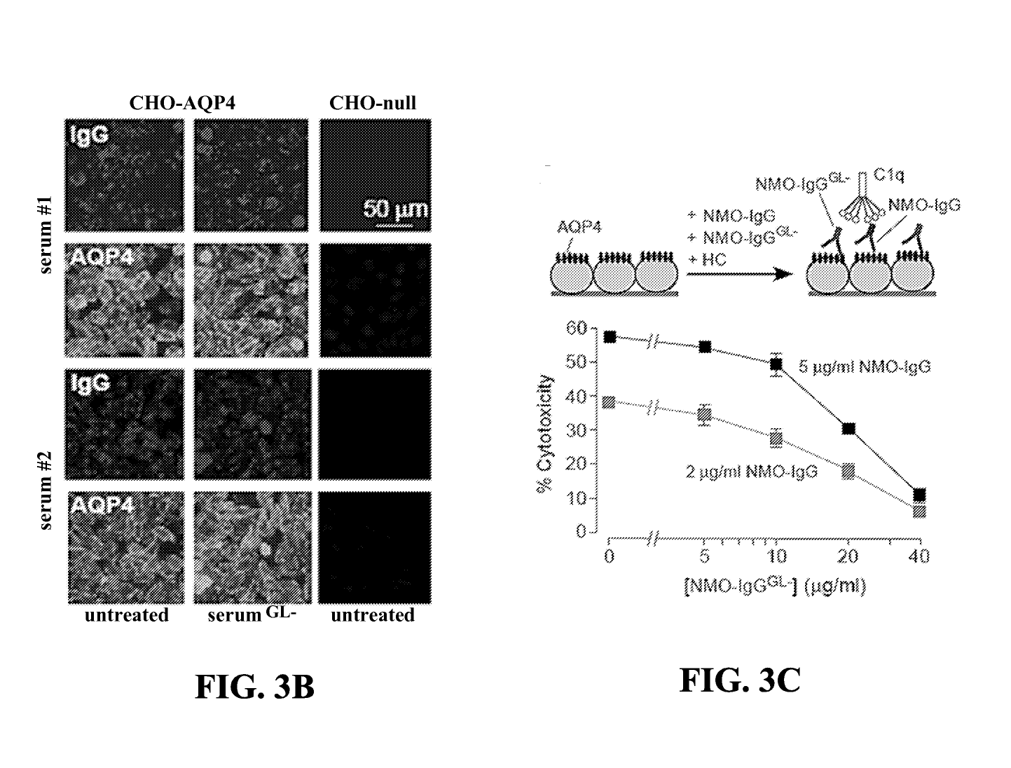

Quantitative measurements were done to determine whether EndoS treatment alters NMO-IgG binding to AQP4, which is expected to depend on the Fab rather than the Fc portion of the antibody. Binding was measured by two-color ratio imaging in which the NMO antibody was stained green (with Alexa-Flour 555-conjugated anti-human secondary antibody) and AQP4 stained red (with anti-C-terminus rabbit primary antibody and Alexa Fluor-488-conjugated anti-rabbit secondary antibody). Fluorescence micrographs in FIG. 3A (left) show saturable antibody binding, with comparable red fluorescence for the control and EndoS-treated antibody. FIG. 3A (right) shows single-site binding curves for control and EndoS-treated NMO-IgG. Antibody binding was not significantly altered by EndoS. Similar measurements in FIG. 3B show that EndoS treatment did not affect AQP4 binding of lgG from serum of two NMO patients.

CDC was measured in AQP4-expressing cells treated with 2 or 5 μg/mL NMO-IgG together with complement and different concentrations of EndoS-treated NMO-IgG. CDC was greatly reduced in a concentration-dependent manner when NMO-IgG was supplemented with excess EndoS-treated NMO-IgG. EndoS-treated NMO antibodies thus function as therapeutic antibodies. As a control, 40 μg/mL EndoS-treated control (non-NMO) antibody did not protect against CDC produced by 2 or 5 μg/mL NMO-IgG.

### Example 7

### Ex Vivo Spinal Cord Slice Model of NMO

Wild type and AQP4 null mice in a CD1 genetic background were used, as generated and characterized previously (Manley et al., (2000) Nat. Med. 6: 159-163). Transverse slices of cervical spinal cord of thickness 300 μm were cut from 7-day old mice using a vibratome and placed in ice-cold Hank's balanced salt solution (HBSS, pH 7.2).

Slices were placed on transparent membrane inserts (Millipore, Millicell-CM 0.4 μm pores, 30 mm diameter) in 6-well plates containing 1 mL culture medium, with a thin film of culture medium covering the slices. Slices were cultured in 5% CO₂ at 37° C. for 7 days in 50% MEM, 25% HBSS, 25% horse serum, 1% penicillin-streptomycin, 0.65% glucose and 25 mM HEPES. On day 7, NMO-IgG (5 μg/mL control or EndoS-treated) and human complement (5%) were added to the culture medium on both sides of the slices. In some experiments NMO-IgG was first added, followed 30 minutes later by EndoS, and 60 minutes thereafter by complement. Slices were cultured for an additional 24 hours, and immunostained for AQP4 and glial fibrillary acid protein (GFAP). Sections were scored as follows: 0, intact slice with normal GFAP and AQP4 staining: 1, mild astrocyte swelling and/or AQP4 staining; 2, at least one lesion with loss of GFAP and AQP4 staining; 3, multiple lesions affecting >30% of slice area; 4, lesions affecting >80% of slice area.

### Example 8

### EndoS Treatment Prevents Lesions in an Ex Vivo Spinal Cord Slice Model of NMO

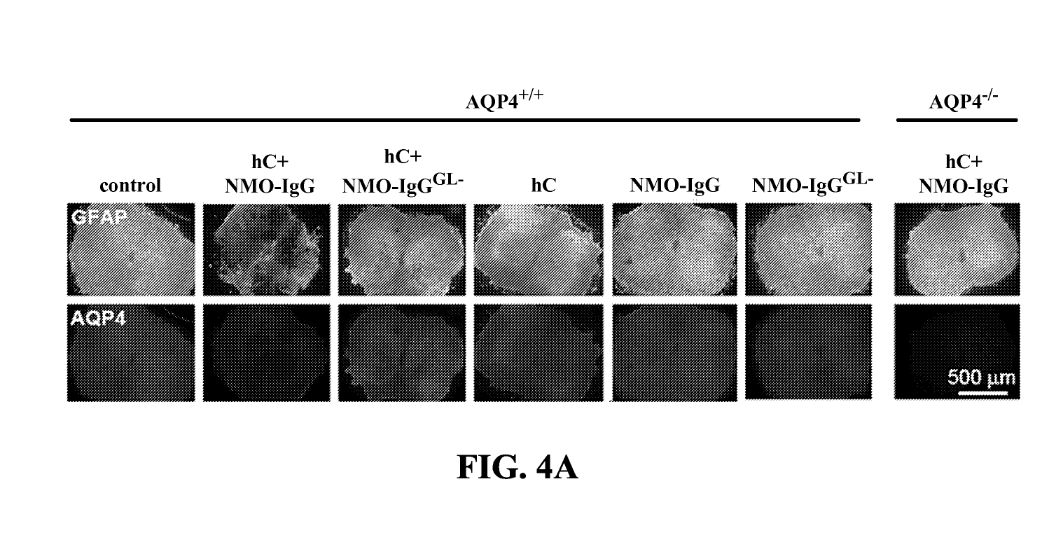

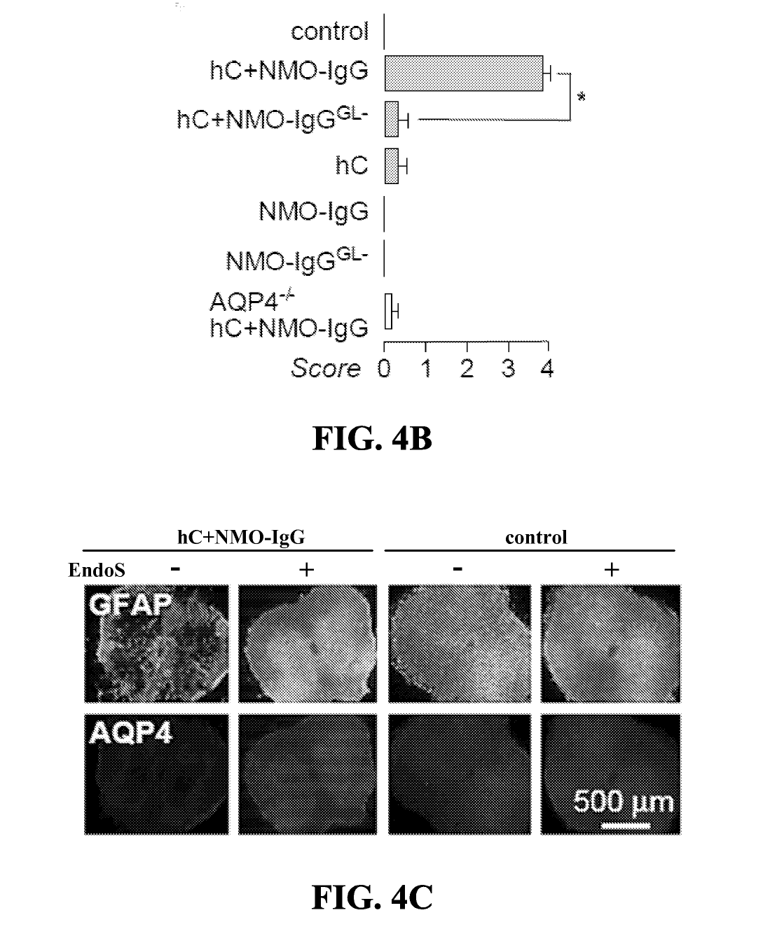

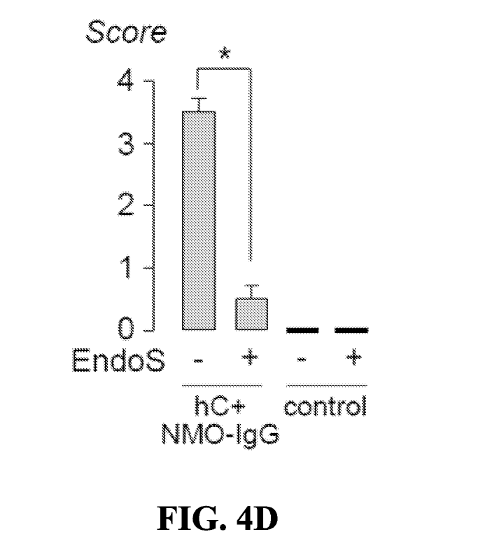

The efficacy of EndoS treatment was tested in a spinal cord slice culture model of NMO, in which NMO-IgG and complement produce lesions with loss of GFAP, AQP4 and myelin, deposition of activated complement, and activation of microglia (Saadoun et al., (2010) Brain 133: 349-361). Spinal cord slices were cultured for 7 days, after which NMO-lgG (control or EndoS-treated) and human complement was added to the culture medium on both sides of the slices. Following an additional 24 hours in culture, slices were immunostained for GFAP and AQP4, and scored for lesion severity. Representative fluorescence micrographs in FIG. 4A and lesion scores in FIG. 4B show marked loss of GFAP and AQP4 in NMO-IgG and complement-treated spinal cord slices, but little loss of GFAP and AQP4 when NMO-IgG was replaced by NMO-IgG^{GL-}. To test the efficacy of EndoS treatment in situ after NMO-IgG binding to AQP4, NMO-IgG was first added to the slices, following by 20 U/mL EndoS 30 minutes later, and then by complement 60 minutes later. Representative fluorescence micrographs in FIG. 4C and lesion scores in FIG. 4D show that EndoS addition post-NMO-IgG prevented lesion development. EndoS alone did not cause damage to the slice cultures.

### Example 9

### In Vivo Mouse Brain Injection Model of NMO

Adult wild type mice (30-35 g) were anesthetized with 2,2,2-tribromoethanol (125 mg/kg i.p.) and mounted in a stereotactic frame. Following a midline scalp incision, a burr hole of diameter 1 mm was made in the skull 2 mm to the right of bregma. A 30-gauge needle attached to 50-μL gas-tight glass syringe (Hamilton) was inserted 3-mm deep to infuse 0.6 μg NMO-IgG (control or EndoS-treated) and 3 μL of human complement in a total volume of 8 μL (at 2 μL/min), as described in Saadoun et al., (2010) Brain 133: 349-361. After 3 days mice were anesthetized and perfused through the left cardiac ventricle with 5 mL PBS and then 20 mL of PBS containing 4% PFA. Brains were post-fixed for 2 hours in 4% PFA. Five-μm-thick paraffin sections were immunostained at room temperature for 1 hour with: rabbit anti-AQP4 (1:200, Santa Cruz Biotechnology, Santa Cruz, Calif.), mouse anti-GFAP (1:100, Millipore, Temecula, Calif.), and goat anti-myelin basic protein (MBP) (1:200, Santa Cruz Biotechnology) followed by the appropriate fluorescent secondary antibody (1:200, Invitrogen). Tissue sections were examined with a Leica DM 4000 B microscope at 25× magnification. AQP4, GFAP and MBP immunonegative areas were defined by hand and quantified using ImageJ.

### Example 10

### EndoS Treatment Prevents Lesions in an In Vivo Mouse Model of NMO

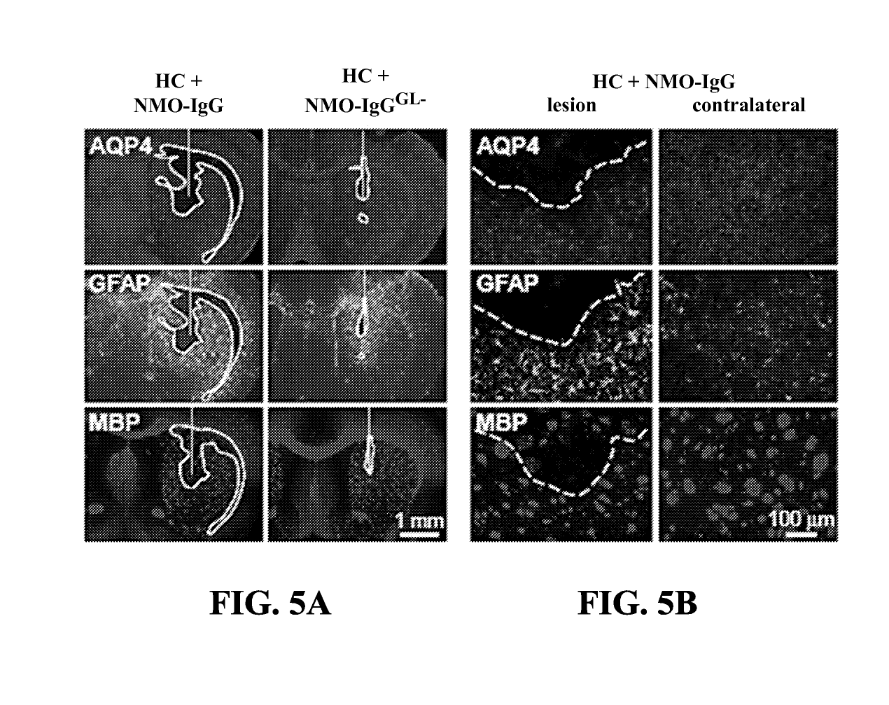

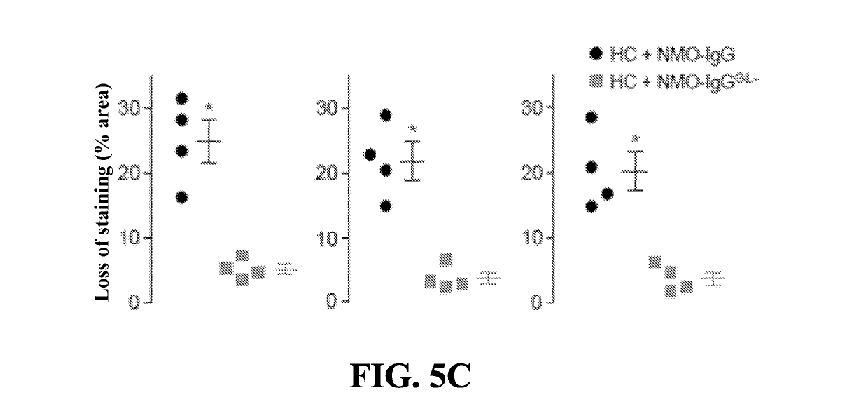

The efficacy of EndoS treatment was also tested in an in vivo mouse model of NMO produced by intracerebral injection of NMO-IgG and human complement (Saadoun et al., (2010) Brain 133: 349-361). FIG. 5A shows marked loss of AQP4, GFAP and myelin in brains of mice injected with NMO-IgG and human complement, in agreement with prior results (Saadoun el al., (2010) Brain 133: 349-361; Saadoun el al., (2012) Ann. Neurol. 71: 323-333). FIG. 5B shows a higher magnification of the lesion with loss of AQP4, GFAP and myelin compared to non-injected contralateral hemisphere. Lesions are surrounded by reactive astrocytes that overexpress the astrocyte marker GFAP (FIG. 5B). Replacement of NMO-IgG with the same concentration of NMO-IgG^{GL-} produced little loss of AQP4, GFAP and myelin (FIG. 5A). Quantification of lesion size showed near absence of astrocyte and oligodendrocyte injury with the EndoS-treated NMO-IgG (FIG. 5C).

### Example 11

### IdeS Cleavage of NMO-IgG Prevents CDC and ADCC

IdeS treatment was performed as follows. IdeS (FabRICATOR®) and IdeS microspin column (FragiT™ Microspin) were purchased from Bulldog Bio Inc. (Rochester, N.Y.). NMO-IgG or NMO serum (or control IgG/serum) was treated by incubation with IdeS (1-5 unit per 1 μg IgG) for up to 1 hour at 37° C.; NMO serum was digested with IdeS using a microspin column containing IdeS covalently coupled to agarose beads. Treated antibody is referred to as NMO-IgGIdeS. Treated NMO serum is referred to as NMO serumIdeS. IdeS treatment was assessed by 10% sodium dodecyl sulfate polyacrylamide gel electrophoresis (SDS-PAGE) followed by staining with Coomassie Blue.

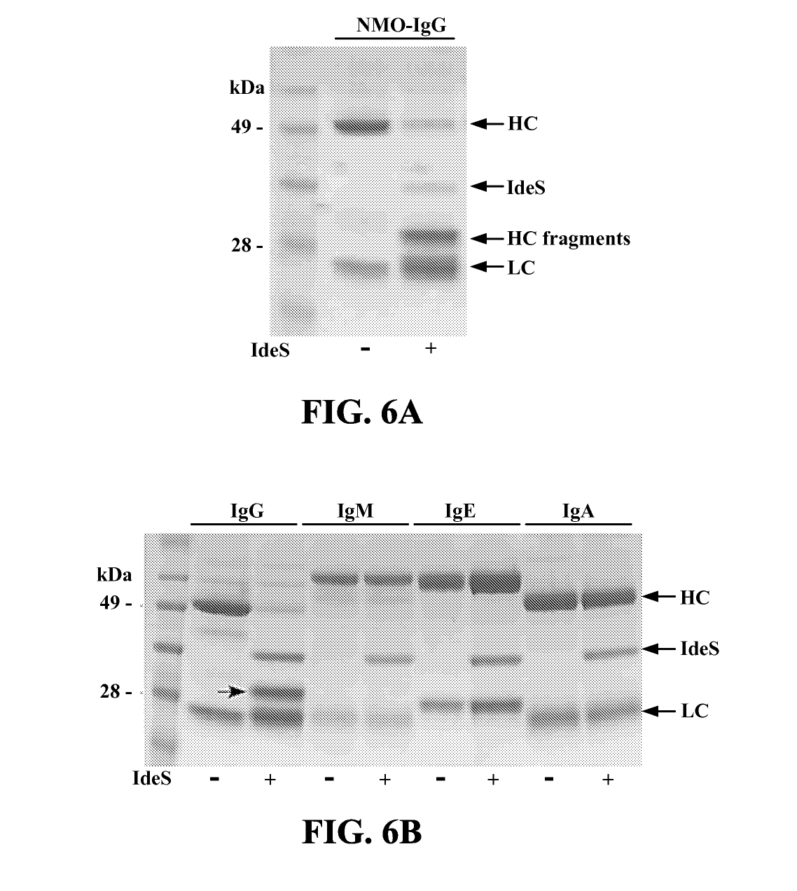

IdeS cleavage of NMO-IgG occurs at the lower hinge/CH₂ (heavy chain constant region 2) region of IgG-class antibody to produce an F(ab′)₂ fragment and two Fc fragments. SDS-PAGE with Coomassie blue staining shows loss of the antibody heavy chain and appearance of smaller fragments following IdeS cleavage of NMO-IgG (FIG. 6A). FIG. 6B verifies the IgG-selective action of IdeS. Purified human IgG, IgM, IgE and IgA were treated with a high concentration of IdeS (5 U IdeS/1 μg immunoglobulin). Whereas cleavage of IgG was essentially complete under these conditions, showing bands at the expected molecular sizes of heavy chain fragments (31 kDa) and light chains (25 kDa), no cleavage was seen for IgM, IgE and IgA.

Because IdeS separates F(ab′)₂ from Fc, it was anticipated that Fc-dependent effector functions of NMO-IgG should be abolished following IdeS cleavage. To accomplish NMO-IgGIdeS binding to cells and subsequent testing of CDC and ADCC effector functions, the following materials and methods were used.

Chinese hamster ovary (CHO) cells stably expressing human M23-AQP4 (Phuan et al., 2012) were cultured at 37° C. in 5% CO₂/95% air in F-12 Ham's Nutrient mix medium supplemented with 10% fetal bovine serum, 200 μg/mL geneticin (selection marker), 100 units/mL penicillin and 100 μg/mL streptomycin. Recombinant monoclonal NMO antibodies rAb-53 and rAb-93 was generated from a clonally expanded plasma blast population from cerebrospinal fluid (CSF) of an NMO patient (Bennett et al., 2009; Crane et al., 2011). NMO serum was obtained from NMO-IgG seropositive individuals who met the revised diagnostic criteria for clinical disease (Wingerchuk et al., 2006). Non-NMO (seronegative) human serum was used as control. For some studies, IgG was purified from NMO or control serum using a protein A resin (GenScript, Piscataway, N.Y.) and concentrated using Amicon Ultra Centrifugal Filter Units (Millipore, Billerica, Mass.). Purified human IgM and IgA were purchased from Calbiochem (San Diego, Calif.), IgE from Abeam (Cambridge, Mass.), and IgG from Thermo Scientific Pierce (Rockford, Ill.).

For NMO-IgGIdeS binding, cells were grown on glass coverslips for 24 hours. After blocking with 1% BSA in PBS, cells were incubated with NMO-IgG or NMO serum (control or IdeS-treated) for 30 minutes at room temperature. Cells were washed with PBS and incubated with Cy3-conjugated AffminiPure goat anti-human IgG, F(ab′)₂ fragment-specific, secondary antibody (1:200, Jackson ImmunoResearch, West Grove, Pa.). For AQP4 immunostaining, cells were fixed in 4% paraformaldehyde (PFA) and permeabilized with 0.2% Triton-X. Rabbit anti-AQP4 antibody (1:200. Santa Cruz Biotech, Dallas, Tex.) was added followed by Alexa Fluor-488 goat anti-rabbit IgG secondary antibody (1:200, Invitrogen, Grand Island, N.Y.) for quantitative ratio image analysis.

To test whether the F(ab′)₂ fragments produced by IdeS cleavage compete with NMO-IgG for binding to AQP4. CHO-M23 cells were plated in black 96-well plates with clear plastic bottom (Corning-Costar) at a density of 25,000 cells per well for 24 hours. After blocking with 1% BSA in PBS, cells were incubated with NMO-IgG and NMO-IgGIdeS or control-IgGIdeS for 30 minutes at room temperature. Cells were washed with PBS and incubated with HRP-conjugated goat anti-human IgG. Fc fragment-specific, secondary antibody (1:500, Invitrogen) for 30 minutes. After washing each well three times with PBS, 50 μl Amplex red substrate (100 μM, Sigma) and 2 mM H₂0₂ were added for measurement of HRP activity. Fluorescence was measured after 45 minutes (excitation 540 nm, emission 590 nm).

For assay of CDC, cells were incubated for 60 minutes at 37° C. with NMO-IgG or NMO serum (control or IdeS-treated) with 2% human complement (Innovative Research, Novi, Mich.). In some experiments NMO-IgG was added 30 minutes before IdeS addition, followed 60 minutes later by complement. Cytotoxicity was measured by the Alamar Blue assay (Invitrogen). For assay of ADCC, NK-92 cells expressing CD16 (Conkwest. San Diego, Calif.) were used as the effector cells. The AQP4-expressing CHO cells were incubated for 1 hour at 37° C. with NMO-IgG and effector cells at an effector:target cell ratio of 4:1. To test the effect of Fc fragments generated by IdeS cleavage on CDC, AQP4-expressing CHO cells were incubated for 1 hour with human IgG Fc fragments (Calbiochem). NMO-IgG (3 μg/ml rAb-53) and 1% human complement. To test the effect on ADCC, human IgG Fc fragments (Calbiochem) were pre-incubated with NK cells for 30 minutes at 37° C., then added together with NMOIgG (3 μg/ml rAb-53) to AQP4-expressing CHO cells and incubated for 1 hour.

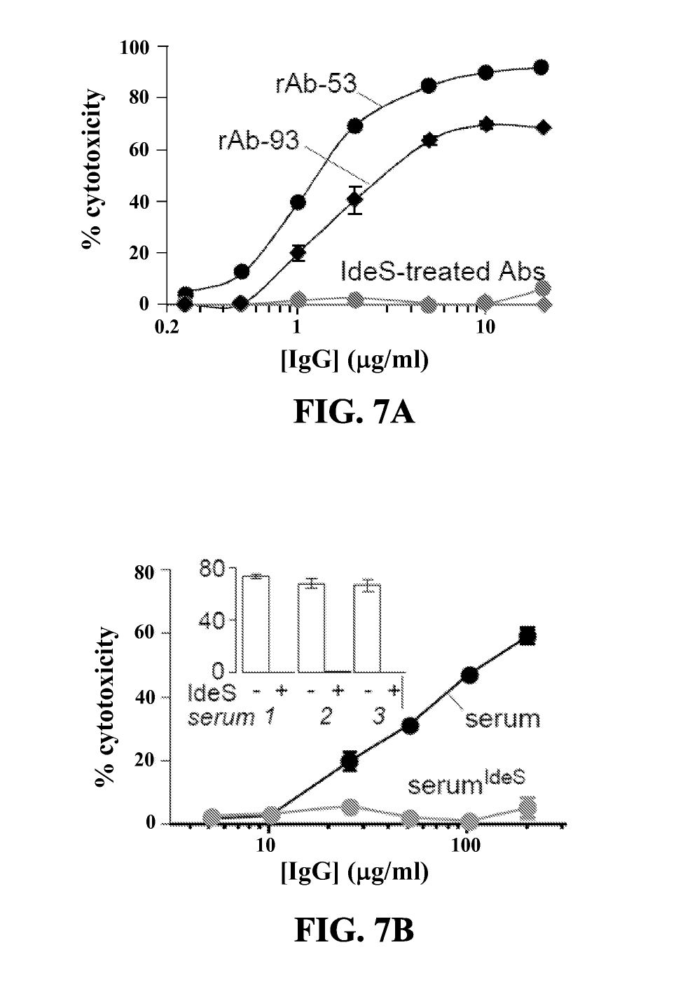

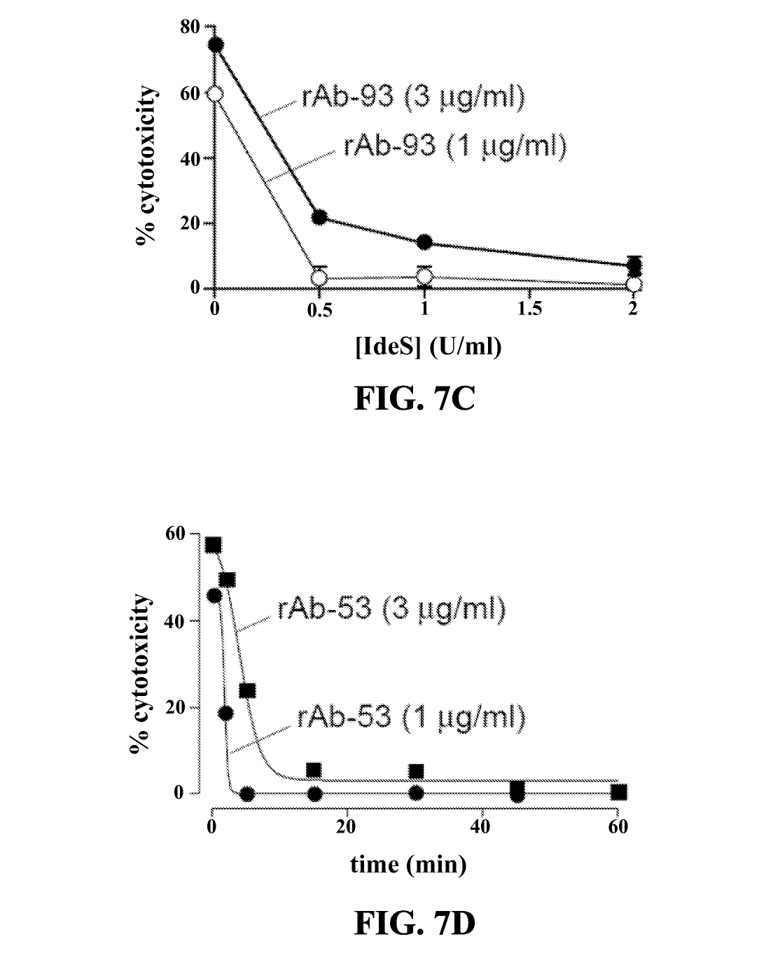

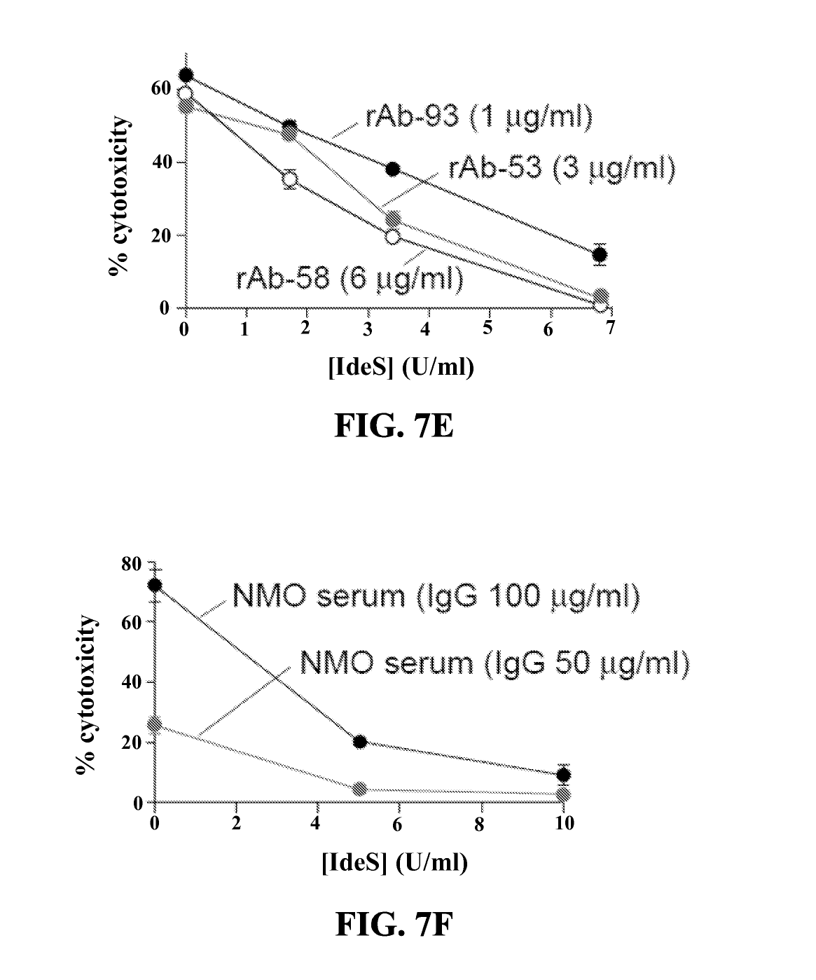

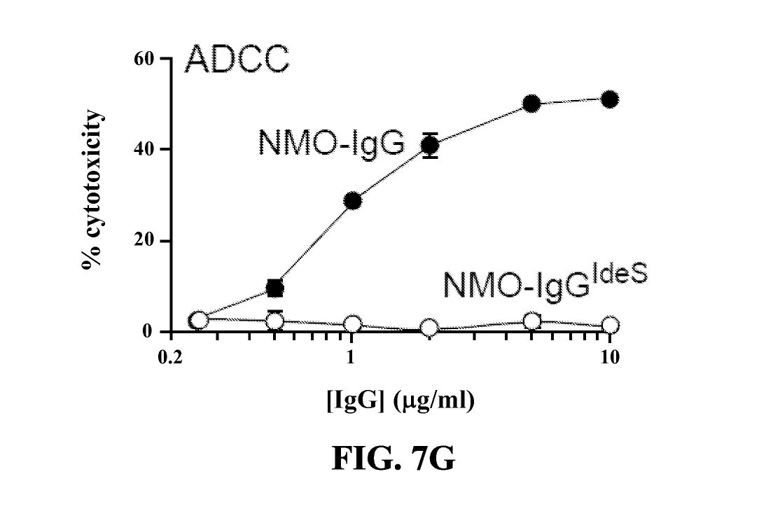

FIGS. 7(A & B) shows loss of CDC, as measured by an Alamar Blue cytotoxicity assay, in AQP4-expressing cells incubated with control or IdeS-treated NMO-IgG, together with human complement. IdeS treatment prevented CDC caused by different monoclonal NMO-IgGs (FIG. 7A) and NMO patient sera (FIG. 7B). FIGS. 7 (C & D) shows the time- and IdeS concentration-dependence for reduction of CDC for two monoclonal NMO-IgGs. FIGS. 7 (E & F) shows that IdeS is effective when NMO-IgG is already bound to AQP4. AQP4-expressing cells were pre-incubated for 30 minutes with NMO-IgG, then IdeS was added, followed 30 minutes later by complement. IdeS treatment after NMO-IgG binding abolished CDC in concentration-dependent manner. FIG. 7G shows that IdeS cleavage abolished the ADCC effector function of NMO-IgG, as demonstrated in a cytotoxicity assay of AQP4-expressing cells incubated with NMO-IgG and human NK-cells.

### Example 12

### IdeS-Cleaved NMO-IgG Binds to AQP4, Competitively Displacing Pathogenic NMO-IgG

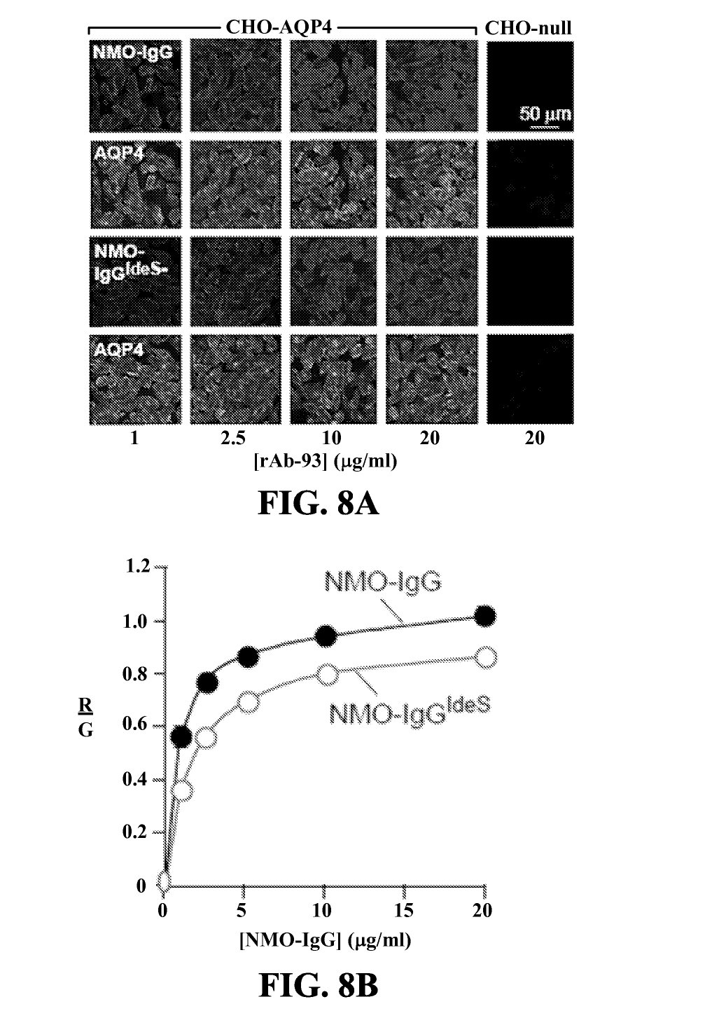

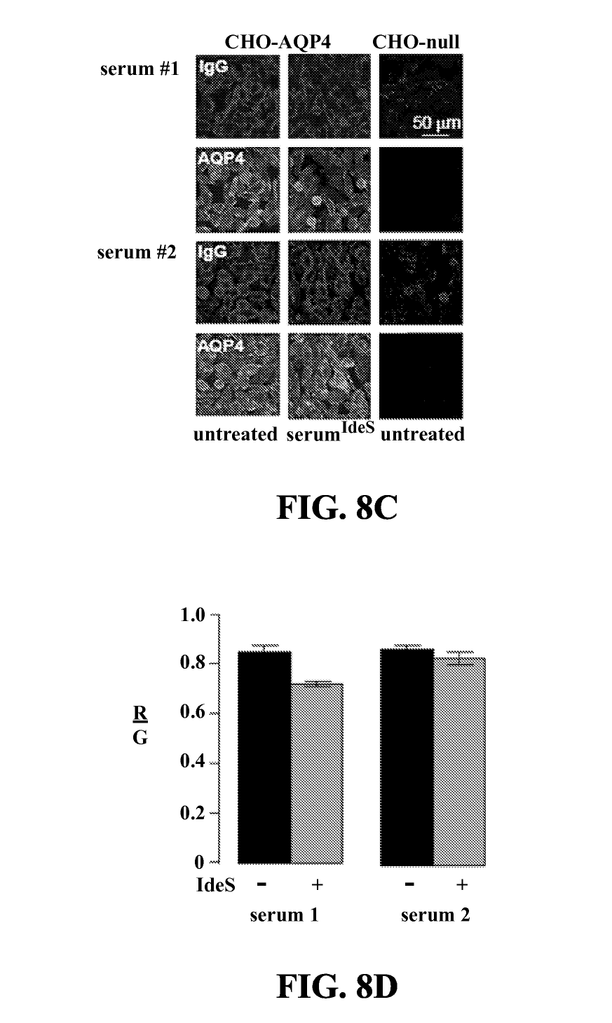

Binding of NMO-IgG to AQP4 was compared with that of the NMO-F(ab′)₂ fragment generated by IdeS cleavage. Binding to AQP4-expressing cells was measured by a ratio imaging assay in which NMO-IgG was stained red (Cy3-conjugated F(ab′)2 fragment-specific anti-human secondary antibody) and AQP4 stained green (anti-C-terminus rabbit primary antibody, Alexa Fluor-488-anti-rabbit secondary antibody). Fluorescence micrographs show similar red fluorescence for control and IdeS-treated NMO-IgG, both for a recombinant NMO-IgG (FIG. 8A) and for NMO patient sera (FIG. 8C). Quantitative ratio image analysis showed little effect of IdeS cleavage on NMO-IgG binding (FIGS. 8B and 8D).

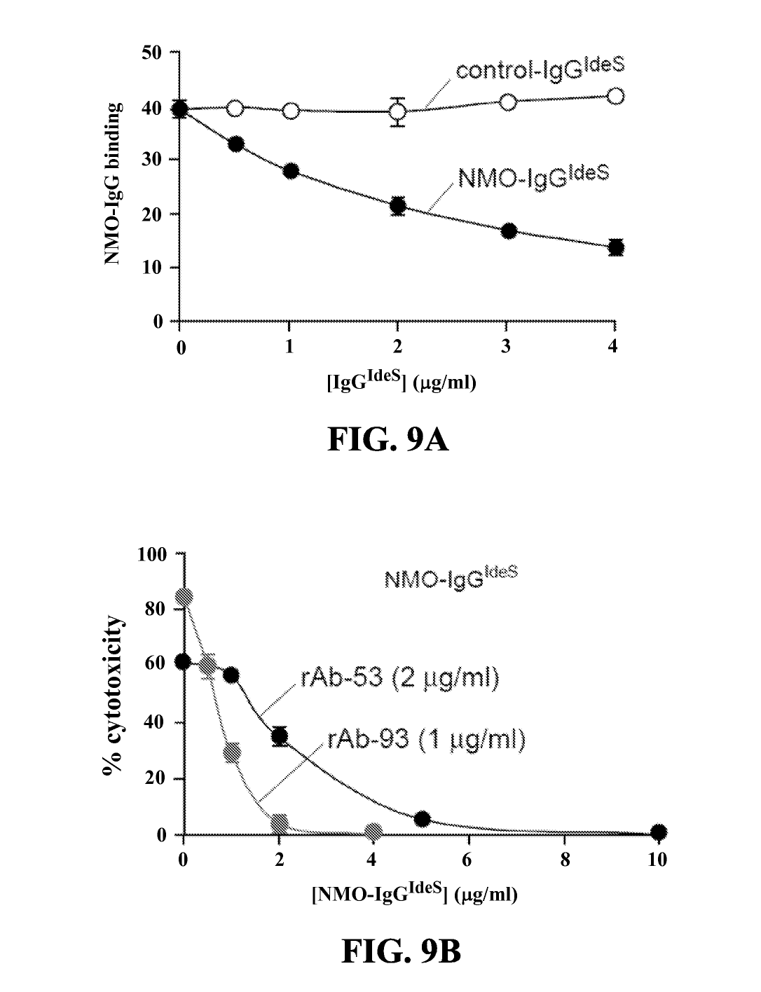

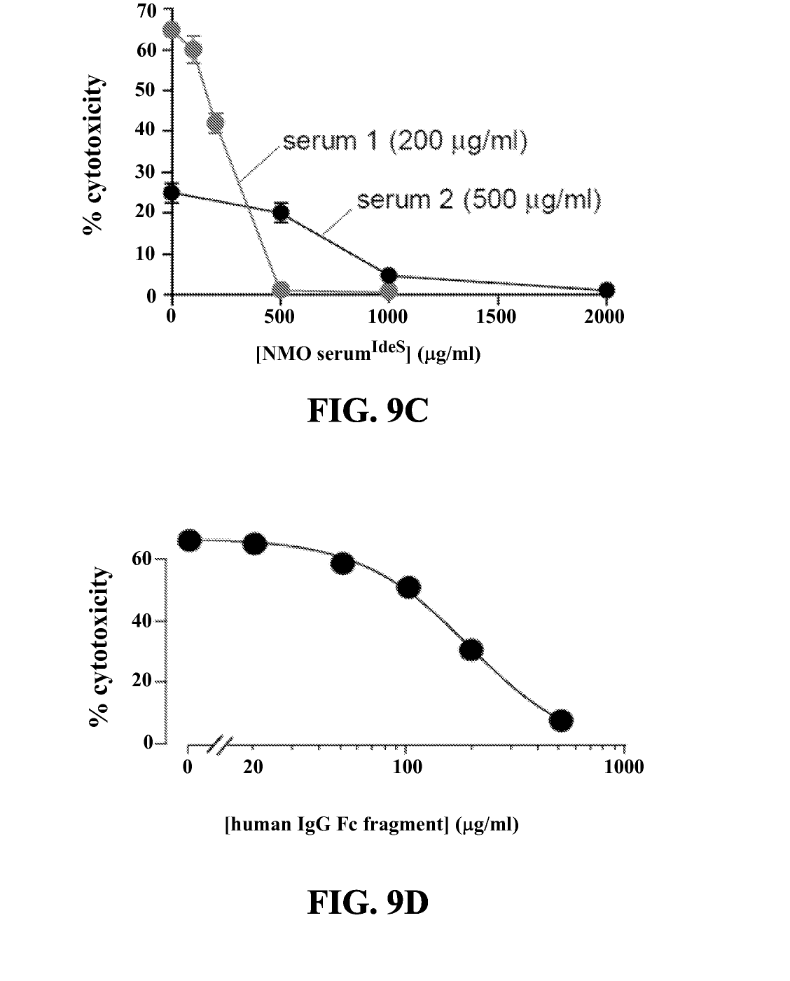

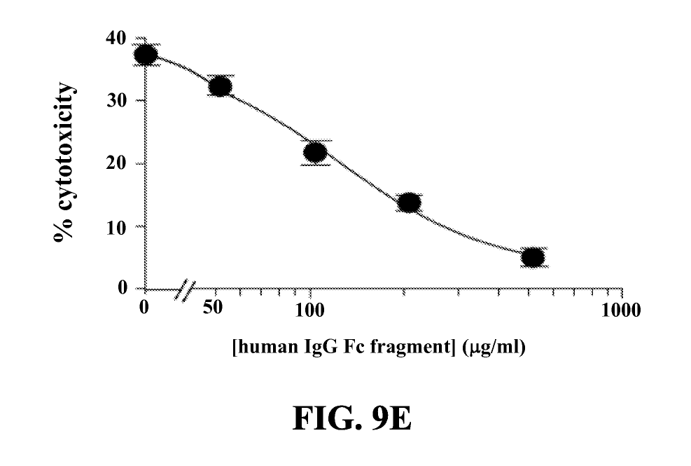

The product NMO-F(ab′)₂ fragments, which lack effector functions, compete with the original NMO-IgG for binding to AQP4. NMO-IgG binding was measured using a horseradish peroxidase-conjugated secondary antibody that recognizes the Fc fragment of the primary antibody (FIG. 9A). NMO-IgG binding was greatly reduced with increasing concentrations of IdeS-treated NMO-IgG (NMO-IgG^{IdeS}), but not of IdeS-treated control antibody (control-IgG^{IdeS}). Also, CDC was measured in AQP4-expressing cells treated with different monoclonal NMO-IgGs or NMO patient sera together with complement, and increasing concentrations of IdeS-treated NMO-IgG. FIGS. 9B and 9C show greatly reduced CDC with increasing concentrations of IdeS-treated NMO-IgG. IdeS cleavage thus converts pathogenic NMO-IgG into non-pathogenic, blocking NMO-F(ab′)₂ fragments that interfere with binding of pathogenic NMO-IgG to AQP4 and downstream cytotoxicity.

### Example 13

### Fc Fragments Released after IdeS Cleavage Reduce CDC and ADCC

To test whether the lgG Fc fragments generated by IdeS can protect against NMO-IgG-induced CDC, AQP4-expressing cells were incubated with NMO-IgG, human complement, and different concentrations of human IgG Fc fragments. CDC was greatly reduced with inclusion of IgG Fc fragments (FIG. 9D). To test whether the IgG Fc fragments protect against NMO-IgG-induced ADCC, increasing concentrations of human IgG Fc fragments were added, together with NMO-IgG and human NK-cells, to AQP4-expressing cells. FIG. 9E shows that IgG Fc fragments prevented NMO-IgG-induced ADCC. The reduced CDC and ADCC is probably related to Fc fragment binding to C1q and Fcγ receptors, respectively.

### Example 14

### IdeS Treatment Reduces NMO Pathology in Mice

IdeS was also tested in a mouse model of NMO produced by intracerebral injection of NMO-IgG and human complement (Saadoun et al., 2010; 2012). The following materials and methods were used in these experiments. Adult wild type mice (30-35 g) were anesthetized with 2,2,2-tribromoethanol (125 mg/kg i.p.) and mounted in a stereotactic frame. Following a midline scalp incision, a burr hole of diameter 1 mm was made in the skull 2 mm to the right of bregma. A 30-gauge needle attached to 50-μL gas-tight glass syringe (Hamilton) was inserted 3-mm deep to infuse 0.6 μg NMO-IgG (control or IdeS-treated) and 3 μL of human complement in a total volume of 8 μL (at 2 μL/min) (Saadoun et al., 2010).

In some experiments, purified IgG from NMO serum (12 μg) was injected together with an excess of IdeS-treated IgG purified from NMO or control serum (48 μg) and 3 μL human complement in a total volume of 18 μL. In some experiments mice were injected with 0.6 μg NMO-IgG and 15 minutes later at the same site with 3 μL human complement with or without 16.75 U IdeS. After 3 days mice were anesthetized and perfused through the left cardiac ventricle with 5 mL PBS and then 20 mL of PBS containing 4% PFA. Brains were post-fixed for 2 hours in 4% PFA. Five μm-thick paraffin sections were immunostained at room temperature for 1 hour with: rabbit anti-AQP4 (1:200, Santa Cruz Biotechnology, Santa Cruz, Calif.), mouse anti-GFAP (1:100, Millipore, Temecula, Calif.), and goat antimyelin basic protein (MBP)(1:200, Santa Cruz Biotechnology) followed by the appropriate fluorescent secondary antibody (1:200, Invitrogen). Tissue sections were photographed using a Leica DM 4000 B fluorescence microscope at 25× magnification. AQP4. GFAP and MBP immunonegative areas were defined by hand and quantified using ImageJ. Data are presented as percentage of immunonegative area (normalized to total area of hemi-brain slice). Protocols were approved by the UCSF Committee on Animal Research.

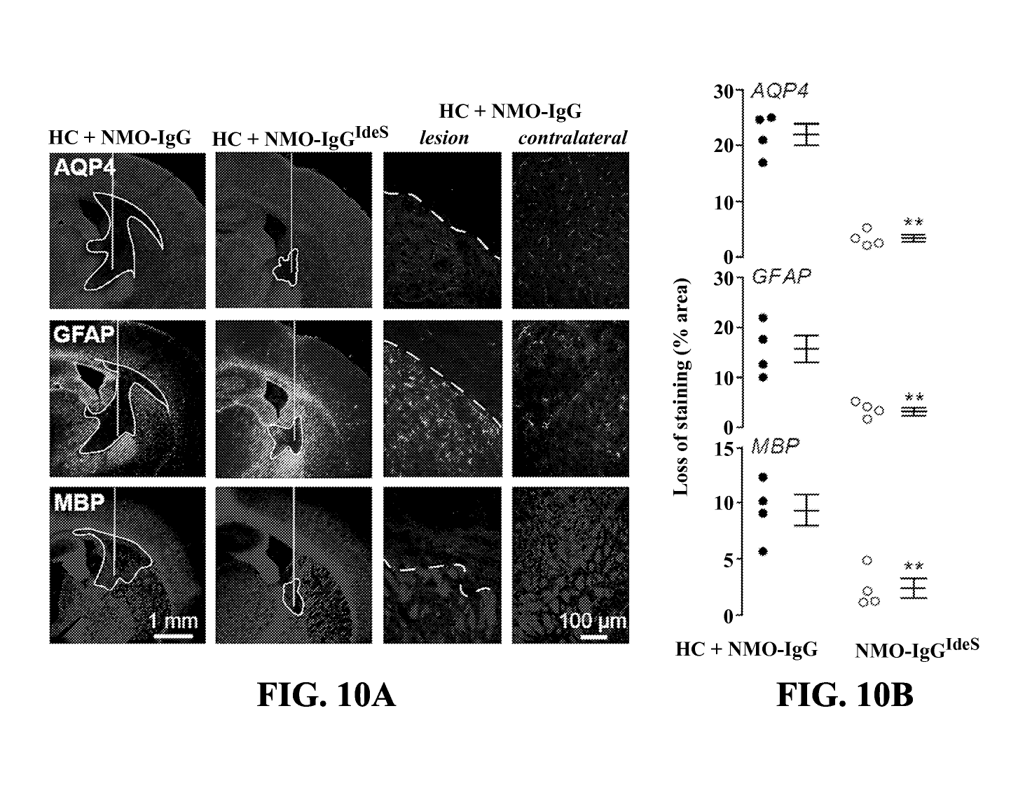

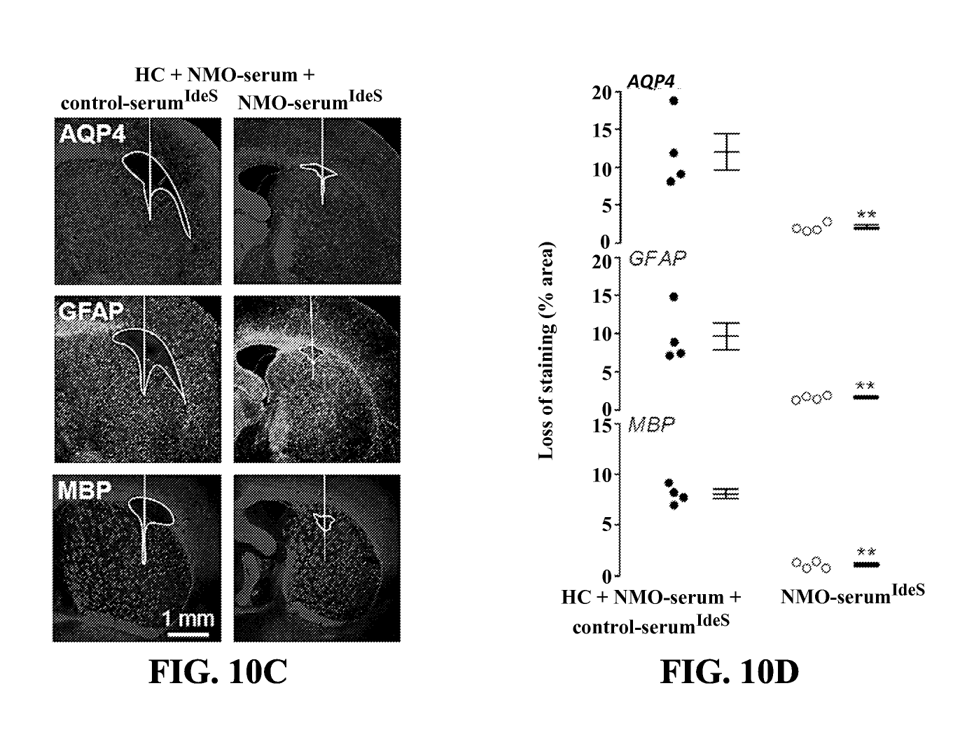

In a first set of studies mice were injected with NMO-IgG (rAb-S3), without or with IdeS pretreatment, together with complement. After 3 days there was marked loss of AQP4, GFAP and myelin around the injection site in mice administered untreated NMO-IgG (FIG. 10A, left), as found previously (Saadoun et al., 2010), with only small lesions in mice receiving IdeS-treated NMO-IgG. Higher magnification of the lesion in mice receiving untreated NMO-IgG shows well-demarcated areas of AQP4, GFAP and myelin loss in the ipsilateral hemisphere, with increased expression of GFAP and AQP4 in reactive astrocytes outside of the lesion (FIG. 10A, right). Loss of GFAP, AQP4 and myelin immunoreactivity was greatly reduced in the mice receiving IdeS-treated NMO-IgG (FIG. 10B).

In a second set of experiments mice were injected with untreated NMO-IgG (purified IgG from NMO patient serum) together with complement, without or with a 4-fold molar excess of IdeS-treated IgG from the same NMO patient. FIG. 10C shows typical lesions in mice receiving untreated NMO-IgG and complement, with much reduced lesion size when excess IdeS-treated NMO-IgG was included. Areas of loss of immunoreactivity are summarized in FIG. 10D. IdeS-treated NMO antibody can thus compete with pathogenic NMO antibody in mouse brain in vivo.

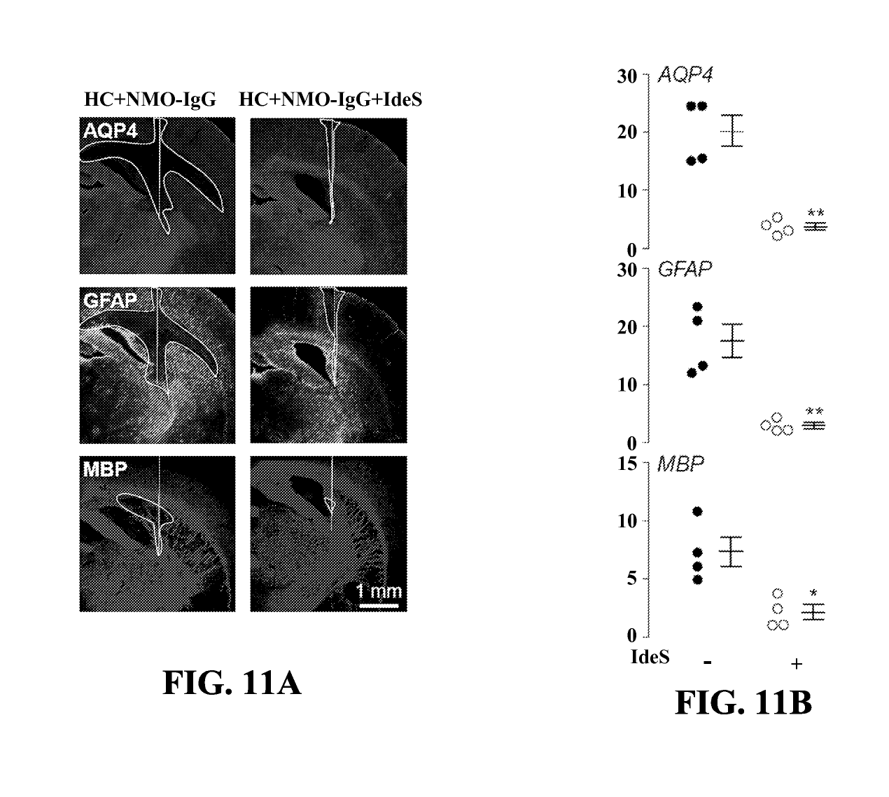

In a third set of in vivo experiments, mice were administered NMO-IgG (rAb-53) followed 15 minutes later by IdeS and complement at the same site. FIG. 11A shows greatly reduced lesion size when IdeS was injected, with a summary of data in FIG. 11B. IdeS can thus cleave NMO-IgG already bound to astrocyte AQP4 in mouse brain in vivo at a sufficiently rapid rate to prevent the development of NMO lesions during exposure to complement.

We claim:

1. A method of treating neuromyelitis optica (NMO) in an animal or human subject comprising administering to the subject a composition comprising a therapeutically effective amount of an Fc region modified anti-AQP4 antibody, thereby treating the NMO in the subject.

2. The method of claim 1, wherein the Fc region modified anti-AQP4 antibody is an anti-AQP4 antibody deglycosylated at the amino acid position Asn297.

3. The method of claim 2, wherein the Fc region modified anti-AQP4 antibody is an immunoglobulin G antibody.

4. The method of claim 2, wherein the Fc region modified anti-AQP4 antibody is administered to the subject intrathecally, intravenously, subdurally, directly to an optic nerve, to the cerebrospinal fluid, or during plasmapharesis.

5. The method of claim 2, wherein the Fc region modified anti-AQP4 antibody is created by treatment of an anti-AQP4 antibody with an Endoglycosidase S.

6. The method of claim 5, wherein the Endoglycosidase S is administered to the subject in an amount effective to create the Fc region modified anti-AQP4 antibody in vivo.

7. The method of claim 6, wherein the Endoglycosidase S is administered to the subject intrathecally, intravenously, subdurally, directly to an optic nerve, to the cerebrospinal fluid, or during plasmapharesis.

8. The method of claim 2, wherein the Fc region modified anti-AQP4 antibody is created using an anti-AQP4 antibody obtained from the subject.

9. The method of claim 1, wherein the Fc region modified anti-AQP4 antibody is an anti-AQP4 antibody F(ab′)₂ fragment.

10. The method of claim 9, wherein the Fc region modified anti-AQP4 antibody is an immunoglobulin G antibody.

11. The method of claim 9, wherein the Fc region modified anti-AQP4 antibody is administered to the subject intrathecally, intravenously, subdurally, directly to an optic nerve, to the cerebrospinal fluid, or during plasmapharesis.

12. The method of claim 9, wherein the Fc region modified anti-AQP4 antibody is created by treatment of an anti-AQP4 antibody with an IdeS enzyme.

13. The method of claim 12, wherein the IdeS enzyme is administered to the subject in an amount effective to create the Fc region modified anti-AQP4 antibody in vivo.

14. The method of claim 13, wherein the IdeS enzyme is administered to the subject intrathecally, intravenously, subdurally, directly to an optic nerve, to the cerebrospinal fluid, or during plasmapharesis.

15. The method of claim 9, wherein the Fc region modified anti-AQP4 antibody is created using an anti-AQP4 antibody obtained from the subject.

16. A composition comprising an isolated therapeutic antibody effective in treating neuromyelitis optica (NMO), wherein said therapeutic antibody is an anti-AQP4 immunoglobulin G deglycosylated at the amino acid position Asn297 thereof.

17. The composition of claim 16, wherein the therapeutic antibody is created by treatment of an anti-AQP4 antibody with an Endoglycosidase S.

18. A composition comprising an isolated therapeutic antibody effective in treating neuromyelitis optica (NMO), wherein said therapeutic antibody is an anti-AQP4 immunoglobulin G F(ab′)₂ fragment.

19. The composition of claim 18, wherein the therapeutic antibody is created by treatment of an anti-AQP4 antibody with an IdeS enzyme.
